(19) United States
(12) Patent Application Publication
(10) Pub. No.: US 2005/0239095 A1
(43) Pub. Date: Oct. 27, 2005

(54) Use of Pin1 inhibitors for treatment of cancer

(75) Inventors: Kun Ping Lu, Newton, MA (US); Janusz M. Sowadski, Boston, MA (US)

Correspondence Address: LAHIVE & COCKFIELD, LLP., 28 STATE STREET, BOSTON, MA, 02109, US

(73) Assignee: BETH ISRAEL DEACONESS MEDICAL CENTER, Boston, MA (US)

(21) Appl. No.: 10/946,445

(22) Filed: Sep. 20, 2004

Related U.S. Application Data
(60) Provisional application No. 60/504,117, filed on Sep. 19, 2003.
(60) Provisional application No. 60/580,814, filed on Jun. 18, 2004.

(51) Int. Cl. C12Q 1/68; G01N 33/574

(52) U.S. Cl. 435/6; 435/7.23

(57) ABSTRACT

The instant invention provides methods for determining if a subject will benefit from treatment with a Pinl modulator based on the expression of Pinl and one or more cancer associated polypeptides, e.g., her2/neu, ras, cyclin Dl, Cdk4, E2F, Myc, Jun, and Rb. The invention further provides methods for determining if a subject will benefit from treatment with one or more cancer treatments, alone or in combination with a Pinl modulator.

### RELATED APPLICATIONS

This application claims the benefit of U.S. Provisional Application 60/504,117, filed on Sep. 19, 2003 and U.S. Provisional Application 60/580,814, filed on Jun. 18, 2004. The contents of the aforementioned applications are hereby expressly incorporated herein by reference.

### GOVERNMENT SUPPORT

This invention was made, in whole or in part, by grants R01GM58556, R01GMAG178870, and R01K08 from the National Institutes of Health. The Government may have certain rights in the invention.

### BACKGROUND

Pinl is a highly conserved protein that catalyzes the isomerization of only phosphorylated Ser/Thr-Pro bonds (Rananathan, R. et al. (1997) Cell 89:875-86; Yaffe, et al. 1997, Science 278:1957-1960; Shen, et al. 1998, Genes Dev. 12:706-720; Lu, et al. 1999, Science 283:1325-1328; Crenshaw, et al. 1998, Embo. J. 17:1315-1327; Lu, et al. 1999, Nature 399:784-788; Zhou, et al. 1999, Cell Mol. Life Sci. 56:788-806). In addition, Pinl contains an N-terminal WW domain, which functions as a phosphorylated Ser/Thre-Pro binding module (Sudol, M. (1996) Prog. Biophys. Mol. Biol. 65:113-32). This phosphorylation-dependent interaction targets Pinl to a subset of phosphorylated substrates, including Cdc25, Wee 1, Myt1, Tau-Rad4, and the C-terminal domain of RNA polymerase II large domain (Crenshaw, D. G., et al. (1998) Embo. J. 17:1315-27; Shen, M. (1998) Genes Dev. 12:706-20; Wells, N. J. (1999) J. Cell. Sci. 112: 3861-71).

The specificity of Pinl activity is essential for cell growth; depletion or mutations of Pinl cause growth arrest, affect cell cycle checkpoints and induce premature mitotic entry, mitotic arrest and apoptosis in human tumor cells, yeast or Xenopus extracts (Lu, et al. 1996, Nature 380:544-547; Winkler, et al. 200, Science 287:1644-1647; Hani, et al. 1999. J. Biol. Chem. 274:108-116). In addition, Pinl is dramatically overexpressed in human cancer samples and the levels of Pinl are correlated with the aggressiveness of tumors. Moreover, inhibition of Pinl by various approaches, including Pinl antisense polynucleotides or genetic depletion, kills human and yeast dividing cells by inducing premature mitotic entry and apoptosis.

Thus, Pinl-catalyzed prolyl isomerization regulates the conformation and function of these phosphoprotein substrates and facilitates dephosphorylation because of the conformational specificity of some phosphatases. Pinl-dependent peptide bond isomerization is a critical post-phosphorylation regulatory mechanism, allowing cells to turn phosphoprotein function on or off with high efficiency and specificity during temporally regulated events, including the cell cycle (Lu et al., supra).

Taken together, these results indicate that the Pin-l subfamily of enzymes is a novel target for diseases characterized by uncontrolled cell proliferation, primarily malignancies. Therefore, there is an ongoing need for specific inhibitors of Pinl and Pinl-related proteins, and for reliable methods of designing such inhibitors. Further, Pinl has been shown to be misexpressed in a large number of cell proliferative disorders (see, for example, WO 02/065091).

### SUMMARY

The present invention is based, at least in part, on the discovery that an animal that is deficient in Pinl expression does not develop cancer when overexpressing a known oncogene.

Accordingly, in one embodiment, the instant invention provides a method of determining if a subject will benefit from treatment with a Pinl inhibitor by obtaining a biological sample from the subject and evaluating the biological sample for the presence of a cancer associated polypeptide, wherein the presence the cancer associated polypeptide indicates that the subject will benefit from treatment with a Pinl inhibitor.

In a related embodiment the biological sample is obtained from a tumor. In another related embodiment, the cancer associated polypeptide is an oncogene. In certain specific embodiments, the oncogene is selected from the group consisting of: her2/neu, ras, cyclin Dl, Cdk4, E2F, Myc, Jun, and Rb. In one particular embodiment, the oncogene is her2/neu. In another particular embodiment, the oncogene is ras.

In specific embodiments the cancer is selected from the group consisting of: breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidney. In one particular embodiment, the subject has a cyclin Dl associated cancer. In one particular embodiment the subject has breast cancer.

In another related embodiment the cancer associated peptide is misexpressed when compared to a control sample.

In one embodiment, the invention provides a method for determining if a subject will benefit from treatment with a cancer associated polypeptide inhibitor by obtaining a biological sample from the subject and evaluating the biological sample for the presence of Pinl wherein an elevated concentration of Pinl in the biological sample indicates that the subject will benefit from treatment with a cancer associated polypeptide inhibitor.

In a related embodiment the biological sample is from a tumor. In a related embodiment the cancer associated polypeptide is encoded by an oncogene.

In certain embodiments the cancer associated polypeptide is selected from the group consisting of: her2/neu, ras, cyclin Dl, Cdk4, E2F, Myc, Jun, and Rb.

In specific embodiments the cancer is selected from the group consisting of: breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidney. In a specific embodiment the cancer is breast cancer.

In a related embodiment, Pinl is misexpressed in the biological sample when compared to a control sample.

In another embodiment, the invention provides a method of determining if a subject will benefit from treatment with a Pinl inhibitor in combination with a second cancer treatment by obtaining a biological sample from a subject and evaluating the biological sample for the presence of Pinl wherein the presence of Pinl is indicative that the subject will benefit from treatment with a Pinl inhibitor and a second cancer treatment specific for the cancer associated polypeptide.

In another embodiment, the invention provides a method of determining if a subject will benefit from treatment with a Pinl inhibitor in combination with a second cancer treatment by obtaining a biological sample from a subject and evaluating the biological sample for the presence of a cancer associated polypeptide wherein the presence of cancer associated polypeptide is indicative that the subject will benefit from treatment with a Pinl inhibitor and a second cancer treatment specific for the cancer associated polypeptide.

In a related embodiment, the method further involves evaluating the biological sample for the presence of a cancer associated polypeptide.

In a related embodiment, the biological sample is from a tumor.

In another related embodiment the cancer associated polypeptide is encoded by an oncogene. In specific embodiments the cancer associated polypeptide is selected from the group consisting of: her2/neu, ras, cyclin Dl, Cdk4, E2F, Myc, Jun, and Rb.

In a particular embodiment the cancer associated polypeptide is her2/neu. In a specific embodiment her2/neu is misexpressed when compared to a control sample.

In another particular embodiment, the cancer associated polypeptide is ras. In a specific embodiment ras is misexpressed when compared to a control sample.

In a further specific embodiment the cancer associated polypeptide is her2/neu and the second cancer treatment is herceptin.

In another embodiment the invention provides a method of treating a subject having a neoplasitic disorder associated with misexpression of a cancer-associated polypeptide by administering to the subject a Pinl inhibitor thereby treating the subject. In a related embodiment, the cancer associated polypeptide is an oncogene. In specific embodiments the cancer associated polypeptide is selected from the group consisting of: her2/neu, ras, cyclin Dl, Cdk4, E2F, Myc, Jun, and Rb. In one specific embodiment, the oncogene is her2/neu.

In a specific embodiment, the neoplasitic disorder associated with over expression of a cancer-associated polypeptide is breast cancer.

In a related embodiment, the subject is administered a her2/neu specific cancer treatment, e.g., herceptin.

In one embodiment the invention provides a method of treating a subject having developed resistance to a first cancer therapy by administering to the subject a Pinl inhibitor thereby treating the subject.

In a related embodiment, the method is useful when a subject developed resistance to a her2/neu specific cancer therapy. In a specific embodiment, the her2/neu specific cancer therapy is herceptin.

In another embodiment, the invention provides a method of treating a subject having a tumor that expresses Pinl and a cancer associated gene comprising administering to the subject a Pinl inhibitor and a second cancer therapy thereby treating the subject having a tumor.

In a related embodiment, the Pinl inhibitor and said second cancer therapy are administered in quantities lower than the quantity that is necessary to be effective if administered alone. In a specific embodiment the second cancer therapy is herceptin.

In a specific embodiment, the subject has cancer, e.g., breast cancer.

In one embodiment, the invention provides a Pinl−/− mouse that is homozygous negative for a cancer associated gene. In a specific embodiment the cancer associated gene is an oncogene.

In another related embodiment, the cancer associated gene is selected from the group consisting of: her2/neu, ras, cyclin Dl, Cdk4, E2F, Myc, Jun, and Rb.

In another embodiment, the invention provides a method of determining the invasive potential of a primary pre-malignant cell comprising the steps of obtaining a biological sample from a subject, isolating cells of interest from the sample, growing the cells on a membrane matrix and analyzing type of growth to thereby determining if a primary cell has invasive potential.

In a related embodiment he method of determines the invasive potential of an epithelial cell. In a further related embodiment, the epithelial cell is isolated from breast tissue.

### BREIF DESCRIPTION OF THE FIGURES

FIG. 1 depicts a graph of survival of Pinl mice that express a Her2/nue transgene as a function of time. The data represents the survival as a function of time for three groups of mice that express the Her2/neu transgene; group 1 is Pinl+/+; group 2 is Pinl±; and group 3 is Pinl−/−.

FIG. 2 depicts a graph of survival of Pinl mice that express a ras transgene as a function of time. The data represents the survival as a function of time for three groups of mice that express the ras transgene; group 1 is Pinl+/+; group 2 is Pinl±; and group 3 is Pinl−/−.

FIG. 3 depicts the results of a three dimensional primary cell differentiation assay. Panel A depicts the number of colonies that have normal morphology in neu wild type and neu knock out mammary epithelial cells. Panel B depicts the number of colonies that have irregular morphology in wild type and knock out mammary epithelial cells. Panel C depicts the number of colonies that have complex structures indicative of invasive growth of infiltrating carcinoma in wild type and knock out mammary epithelial cells.

FIGS. 4A-E depict expression of Pinl and transgenes in mammary glands from normal and cancer tissues derived from the crossbreeding. (A-C) Pinl protein is absent in Pinl-deficient (Pinl−/−) mice (A), but remains at Pinl+/+ levels in Pinl heterozygote (Pinl±) mice (B). Mammary glands and breast cancer tissues from littermates with indicated genotypes were homogenized and equal amounts of total protein were separated on SDS-containing gels and transferred to membranes. The membranes were cut into two pieces and subjected to immunoblotting analysis with antibodies against to Pinl and tubulin (A, B), followed by semi-quantified using Imagequant. The Pinl/tubulin ratio was obtained for mammary glands from 4 different animals and presented in (C). Note that Pinl levels in c-Neu or Ha-Ras transgenic mice are variable, but generally higher than in non-transgenic mice (A, C). There was no statistically significant difference in Pinl levels between Pinl+/+ and Pinl± mice. (D, E). Pinl ablation does not affect the expression of the transgenes Ha-Ras or c-Neu. Protein lysates or tissue sections of mammary glands of the specified genotypes were subjected to immunoblotting (D) or immunostained (E) with anti-c-Neu or anti-Ha-Ras antibodies. Note that out of 3-5 mice analyzed each group, there was no statistically significant difference in Neu or Ras levels between Pinl+/+ and Pinl−/− mice.

FIGS. 5A-C indicate that Pinl ablation is highly effective in preventing breast cancers induced by MMTV-Neu or -Ras, but not -Myc. Transgenic mice overexpressing activated c-Neu, Ras or Myc (FIGS. 5A, B, and C, respectively) under the control of the promoter MMTV were crossbred with Pinl−/− mice to generate mice with nine different genotypes. Virgin females were observed for 75 weeks. Breast cancers were recorded at the time of first palpation.

FIGS. 6A-B indicate that Pinl ablation effectively blocks the induction of cyclin Dl by Neu or Ras. Protein lysates or tissue sections of mammary glands from virgin littermates of the specified genotypes were subjected to immunoprecipitation with anti-cyclin Dl or control IgG, followed by immunoblotting with anti-cyclin Dl antibodies (A) or to immunohistochemistry with anti cyclin Dl antibodies (B).

FIGS. 7A-H indicate that Pinl ablation does not affect the differentiation of primary MECs in 3D cultures. Primary MECs were isolated from morphologically and histologically normal mammary glands of non-transgenic or Neu transgenic littermates in Pinl+/+ or Pinl−/− background at ages of 3-4 months. After culture in collagen-coated plates for 3-5 days, MECs were plated as single cell suspension in reconstituted basement membrane (Matrigel) and analyzed at the indicated time points. Phase images were taken on 5,10 and 20 days in culture, followed by fixation and confocal immunofluorescence staining with anti-E-cadherin antibodies. FIG. 7A depicts phase images of Pin+/+ MECs at 5, 10 and 20 days. FIG. 7B depicts phase images of Pinl−/− MECs at 5, 10, and 20 days. FIG. 7C depicts Pinl+/+ confocal immunofluorescence stained images at 5, 10 and 20 days. FIG. 7D depicts Pinl−/− confocal immunofluorescence stained images at 5, 10 and 20 days. FIG. 7E depicts phase images of Neu/Pin+/+ MECs at 5, 10 and 20 days. FIG. 7F depicts phase images of Neu/Pinl−/− MECs at 5, 10, and 20 days. FIG. 7G depicts Neu/Pinl+/+ confocal immunofluorescence stained images at 5, 10 and 20 days. FIG. 7H depicts Neu/Pinl−/− confocal immunofluorescence stained images at 5, 10 and 20 days.

FIGS. 8A-H depict the characterization of abnormal differentiation patterns of MECs derived from Neu or Ras transgenic mice in Pinl+/+ or Pinl−/− genetic background. Primary MECs were isolated from littermates with different genetic background and subjected to 3D cultures in reconstituted basement membrane for 20 days. Colonies were analyzed by phase contrast microcopy to reveal the morphology (A, F), fixed and stained with hematoxylin and eosin to reveal the histology (B, G), stained with anti-E-cadherin antibodies to reveal the cell polarity (C, H), with anti-a6 integrin to reveal the base membrane integrity (D), with anti-Ki67 antibodies to reveal cell proliferation (E). Based on these assays, colonies are divided into three categories, namely “Regular”, “Irregular” and “Cancer-like”. Arrows in (A, F) point to cell surface spikes protruding into the Matrigel, while an arrows in (B) points to a dividing cell. (A, B, F, G) Light microscopy at 200×; (C-E, H), confocal fluorescence microscopy at 200×.

FIGS. 9A-F depict non-neoplastic primary MECs of Neu or Ras mice in the Pinl+/+, but not Pinl−/− background exhibit the malignant phenotype, including forming tumors in nude mice. (A-D) Primary MECs were isolated from littermates with different genetic background and subjected to 3D cultures in reconstituted basement membrane for 20 days. Assays were set up for 3 to 5 mice of each genotype, plated in quadruples. Colonies were categorized and counted under phase microscopy. The number of colonies in different categories per 10,000 cells plated was plotted as mean ±SD, with p values being indicated. N.S., not significant. (E) Secondary colony formation. “Regular” and “Irregular” colonies derived from Neu or Ras MECs in Pinl+/+ or Pinl−/− background in 3D cultures were picked separately at 21 days and trypsinized, followed by a more round of 3D cultures for 20 days. (F) MEC colonies derived from Neu transgenic mice only in Pinl+/+, but not Piril−/− background give rise to tumors in nude mice. Day 21 colonies were harvested and resuspended in 100 ul MEGM/4% Matrigel, followed by injecting subcutaneously into female nude mice in duplicates each (right and left flank). Out of 6 injections of three mice each group, three tumors were derived from Neu/Pinl+/+ colonies, but Neu/Pinl−/− colonies did not generate any tumors.

FIGS. 10A-C depict expression of cyclin Dl or its T286A mutant restores the malignant phenotype of Neu/Pinl−/− primary MECs. Primary MECs derived from Neu/Pinl−/− mice were infected with retroviruses for either control, cyclin Dl or cyclin Dl^{T286A}, followed by 3D culture on Matrigel. Expression of cyclin Dl in infected MECs was monitored by Western Blotting. At day 21, colonies were analyzed by phase contrast microcopy to reveal the morphology (A), fixed and stained with anti-a6 integrin antibodies to assay basement membrane integrity (B). Colonies were categorized and counted under phase microscopy (C).

FIG. 11 depicts a table of breast cancer incidence of transgenic mice in different Pinl backgrounds (Table 1).

FIG. 12 depicts a table indicating that Pinl does not affect the development of virgin mammary glands (Table 2).

### DETAILED DESCRIPTION

The instant invention is based, at least in part, on the discovery that mice that are deficient in Pinl expression are protected from developing cancer, e.g., breast cancer when over expressing a cancer associated gene, e.g., an oncogene.

I. Definitions

The term “cancer associated polypeptide” refers to a polypeptide whose misexpression has been shown to cause, or be associated with aberrant cell growth, e.g., cancer. Further, cancer associated polypeptides are those that are differentially expressed in cancer cells. In one embodiment, the cancer associated polypeptide is encoded by an oncogene. In a related embodiment, the cancer associated polypeptide is a polypeptide whose expression has been linked to cancer, e.g., as a marker. The presence of a cancer associated polypeptide can be determined by the presence of the polypeptide or nucleic acid molecules, e.g., mRNA or genomic DNA, that encodes the cancer associated polypeptide. Exemplary cancer associated polypeptides include the protein encoded by Her2/neu, (c-erb-2) (Liu et al. (1992) Oncogene 7:1027-32); ras (Nakano, et al. (1984) Proc. Natl. Acad. Sci. U.S.A 81:71-5); Cyclin Dl (Bartkova, et al. (1995) Oncogene 10:775-8, Shamma, et al. (1998) Int. J. Oncol. 13:455-60); E2F1 (Johnson et al. (1994) Proc. Natl. Acad. Sci. 91:12823-7); myc (Corcoran et al. (1984) Cell 37:113-22, Goddard et al. (1986) Nature 322:555-557); jun (Vogt et al. (1990) Adv. Cancer Res. 55:1-35); p53 (Levine et al. (1989) Princess Takamatsu Symp. 20:221-230).

The language “Pinl modulating compound” refers to compounds that modulate, e.g., inhibit, promote, or otherwise alter, the activity of Pinl. Pinl modulating compounds include both Pinl agonists and antagonists. In certain embodiments, the Pinl modulating compound induces a Pinl inhibited-state. In certain embodiments, the Pinl modulating compounds include compounds that interact with the PPI and/or the WW domain of Pinl. In certain embodiments, the Pinl modulating compound is substantially specific to Pinl. The phrase “substantially specific for Pinl” is intended to include inhibitors of the invention that have a Kᵢ or K_{d} that is at least 2, 3, 4, 5, 10, 15, or 20 times less than the Kᵢ or K_{d} for other peptidyl prolyl isomerases, e.g., hCyP-A, hCyP-B, hCyP-C, NKCA, hFKBP-12, hFKBP-13, and hFKBP-25.

In one embodiment of the invention, the Pinl modulating compound of the invention is capable of chemically interacting with Cys113 of Pinl. The language “chemical interaction” is intended to include, but is not limited to reversible interactions such as hydrophobic/hydrophilic, ionic (e.g., coulombic attraction/repulsion, ion-dipole, charge-transfer), covalent bonding, Van der Waals, and hydrogen bonding. In certain embodiments, the chemical interaction is a reversible Michael addition. In a specific embodiment, the Michael addition involves, at least in part, the formation of a covalent bond.

The language “Pinl inhibiting compound” includes compounds that reduce or inhibit the activity of Pinl. In certain embodiments, the Pinl inhibiting compounds include compounds that interact with the PPI and/or the WW domain of Pinl.

In certain embodiments the inhibitors have a Kᵢ for Pinl of less than 0.2 mM, less than 0.1 mM, less than 750 μM, less than 500 μM, less than 250 μM, less than 100 μM, less than 50 μM, less than 500 nM, less than 250 nM, less than 50 nM, less than 10 nM, less than 5 nM, or or less than 2 nM.

The term “Pinl inhibitor” refers to any molecule that can interact with Pinl or a Pinl-related polypeptide and inhibit the ability of the polypeptide to carry out proline isomerization activity. Compounds within the scope of the invention can be naturally occurring or chemically synthesized. The term is also intended to include pharmaceutically acceptable salts of the compounds. In certain embodiments, the inhibitor is specific for Pinl, i.e., does not inhibit the isomerase activity of PPIases belonging to other classes (e.g., cyclophilins or FKBPs).

As used herein, the term “misexpression” includes a non-wild type pattern of gene expression. Expression as used herein includes transcriptional, post transcriptional, e.g., mRNA stability, translational, and post translational stages. Misexpression includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. Misexpression includes any expression from a transgenic nucleic acid. Misexpression includes the lack or non-expression of a gene or transgene, e.g., that can be induced by a deletion of all or part of the gene or its control sequences.

As used herein, the term “knockout” refers to an animal or cells therefrom, in which the insertion of a transgene disrupts an endogenous gene in the animal or cell therefrom. This disruption can essentially eliminate Pinl in the animal or cell.

As used herein, the term “abnormal cell growth” is intended to include cell growth which is undesirable or inappropriate. Abnormal cell growth also includes proliferation which is undesirable or inappropriate (e.g., unregulated cell proliferation or undesirably rapid cell proliferation). Abnormal cell growth can be benign and result in benign masses of tissue or cells, or benign tumors. Many art-recognized conditions are associated with such benign masses or benign tumors including diabetic retinopathy, retrolental fibrioplasia, neovascular glaucoma, psoriasis, angiofibromas, rheumatoid arthritis, hmangiomas, and Karposi's sarcoma. Abnormal cell growth can also be malignant and result in malignancies, malignant masses of tissue or cells, or malignant tumors. Many art-recognized conditions and disorders are associated with malignancies, malignant masses, and malignant tumors.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to grow and function somewhat independently of the homeostatic mechanisms which control normal tissue growth and function. However, some neoplasms remain under the control of the homeostatic mechanisms which control normal tissue growth and function. For example, some neoplasms are estrogen sensitive and can be arrested by anti-estrogen therapy. Neoplasms can range in size from less than 1 cm to over 6 inches in diameter. A neoplasm even 1 cm in diameter can cause biliary obstructions and jaundice if it arises in and obstructs the ampulla of Vater.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histologic and others features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly than malignant tumors. Benign neoplasms also tend to be less autonomous than malignant tumors. Benign neoplasms tend to closely histologically resemble the tissue from which they originated. More highly differentiated cancers, cancers that resemble the tissue from which they originated, tend to have a better prognosis than poorly differentiated cancers. Malignant tumors are more likely than benign tumors to have aberrant functions (i.e. the secretion of abnormal or excessive quantities of hormones).

As used herein, the term “cancer” includes a malignancy characterized by deregulated or uncontrolled cell growth, for instance carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

“Inhibiting tumor growth” or “inhibiting neoplasia” is intended to include the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. Most preferably the subject is a human.

The language “effective amount” of the compound is that amount necessary or sufficient to treat or prevent a subject from developing cancer or from the cancer progressing in a subject that has already developed cancer. In an example, an effective amount of an inhibitor of the invention is the amount sufficient to inhibit undesirable cell growth in a subject. In another example, an effective amount of the inhibitor compound is the amount sufficient to reduce the size of a pre-existing benign cell mass or malignant tumor in a subject. The effective amount can vary depending on such factors as the size and weight of the subject, the type of illness, or the particular compound. For example, the choice of the inhibitor can affect what constitutes an “effective amount”. One of ordinary skill in the art would be able to study the aforementioned factors and make the determination regarding the effective amount of the Pinl binding compound without undue experimentation. In one possible assay, an effective amount of an inhibitor compound can be determined by assaying for the expression of a cancer associated polypeptide and determining the amount of the cancer associated polypeptide inhibitor sufficient to reduce the levels of cancer associated polypeptide to that associated with a non-cancerous state.

The regimen of administration can affect what constitutes an effective amount. The inhibitor compound can be administered to the subject either prior to or after the onset of cancer. Further, several divided dosages, as well as staggered dosages, can be administered daily or sequentially, or the dose can be continuously infused, or can be a bolus injection. Further, the dosages of the Pinl inhibitor(s) can be proportionally increased or decreased as indicated by the exigencies of the therapeutic or prophylactic situation.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

The language “radiation therapy” includes the application of a genetically and somatically safe level of electrons, protons, or photons, both localized and non-localized, to a subject to inhibit, reduce, or prevent symptoms or conditions associated with undesirable cell growth. The term X-rays is also intended to include machine-generated radiation, clinically acceptable radioactive elements, and isotopes thereof, as well as the radioactive emissions therefrom. Examples of the types of emissions include alpha rays, beta rays including hard betas, high-energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases.

“Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment according to that individual's drug response genotype.

Information generated from pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a Pinl molecule or Pinl modulator, such as a modulator identified by one of the exemplary screening assays described herein.

The term “metastasis” as used herein refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

“Invasive” or “aggressive” as used herein with respect to cancer refers to the proclivity of a tumor for expanding beyond its boundaries into adjacent tissue, or to the characteristic of the tumor with respect to metastasis (Darnell, J. (1990), Molecular Cell Biology, Third Ed., W.H.Freeman, NY). Invasive cancer can be contrasted with organ-confined cancer. For example, a basal cell carcinoma of the skin is a non-invasive or minimally invasive tumor, confined to the site of the primary tumor and expanding in size, but not metastasizing. In contrast, the cancer melanoma is highly invasive of adjacent and distal tissues. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

“Biological samples” include solid and body fluid samples. The biological samples of the present invention may include cells, protein or membrane extracts of cells, blood or biological fluids such as ascites fluid or brain fluid (e.g., cerebrospinal fluid). Examples of solid biological samples include samples taken from feces, the rectum, central nervous system, bone, breast tissue, renal tissue, the uterine cervix, the endometrium, the head/neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of “body fluid samples” include samples taken from the blood, serum, semen, prostate fluid, seminal fluid, urine, saliva, sputum, mucus, bone marrow, lymph, and tears. For amplifying RNA, the preferred samples include peripheral venous blood samples and tissue samples. Samples for use in the assays of the invention can be obtained by standard methods including venous puncture and surgical biopsy. In one embodiment, the biological sample is a breast tissue sample obtained by needle biopsy.

II. Diagnostic Methods

As described in more detail below, the detection methods of the invention can be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations, immunohistochemistry and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques. Nucleic acid probes as well as antibodies to Pinl for use in these methods can readily be designed since the nucleic and amino acid sequence of Pinl is known (Hunter et al., WO 97/17986 (1997); Hunter et al., U.S. Pat. Nos. 5,952,467 and 5,972,697).

Methods of detecting specific nucleic acid molecules or polypeptides in a biological sample are known in the art. Specific examples of methods of detecting cancer associated polypeptides are described in U.S. Pat. No. 6,512,097 which describes antibodies to c-erbB-2 protein product of the HER2/neu oncogene; U.S. Pat. No. 5,262,523 which describes antibodies reactive with normal and oncogenic forms of the ras p21 protein; and U.S. Pat. No. 6,025,151 which describes the measurement of c-fos and c-jun mRNA by Northern analysis using specific oligomers. The methods described in the aforementioned patents can be modified to detect the presence of other cancer associated polypeptides and nucleic acid molecules as described below.

The diagnostic methods described herein can be used to determine if a subject will benefit from treatment with a Pinl inhibitor or a combination of a Pinl inhibitor in and a cancer associated polypeptide inhibitor.

A. Antibody-Based Assays

In embodiments of the methods disclosed herein, assessing the level of Pinl or a cancer associated polypeptide in a biological sample from the subject includes contacting the biological sample with an antibody to Pinl, a cancer associated polypeptide, or a fragment thereof; determining the amount of binding of the antibody to the biological sample; and comparing the amount of antibody bound to the biological sample to a predetermined base level.

The level of Pinl and/or a cancer associated polypeptide in normal (i.e. non-cancerous) biological samples can be assessed in a variety of ways. In one embodiment, this normal level of expression is determined by assessing the level of Pinl or a cancer associated polypeptide in a portion of cells which appears to be non-cancerous and by comparing this normal level with the level of Pin-1 in a portion of the cells which is suspected of being cancerous. Alternatively, the ‘normal’ level of expression of a marker may be determined by assessing the level of Pinl or a cancer associated polypeptide in a sample or samples obtained from a non-cancer-afflicted individuals.

“Antibody” includes immunoglobulin molecules and immunologically active determinants of immunoglobulin molecules, i.e., molecules that contain an antigen binding site which specifically binds (immunoreacts with) an antigen. Structurally, the simplest naturally occurring antibody (e.g., IgG) comprises four polypeptide chains, two copies of a heavy (H) chain and two of a light (L) chain, all covalently linked by disulfide bonds. Specificity of binding in the large and diverse set of antibodies is found in the variable (V) determinant of the H and L chains; regions of the molecules that are primarily structural are constant (C) in this set. Antibody includes polyclonal antibodies, monoclonal antibodies, whole immunoglobulins, and antigen binding fragments of the immunoglobulins. Pinl specific antibodies are described in U.S. Pat. No. 6,596,848.

The binding sites of the proteins that comprise an antibody, i.e., the antigen-binding functions of the antibody, are localized by analysis of fragments of a naturally-occurring antibody. Thus, antigen-binding fragments are also intended to be designated by the term “antibody.” Examples of binding fragments encompassed within the term antibody include: a Fab fragment consisting of the V_{L}, V_{H}, C_{L} and C_{H1} domains; an F_{d} fragment consisting of the V_{H} and C_{H1} domains; an Fᵥ fragment consisting of the V_{L} and V_{H} domains of a single arm of an antibody; a dAb fragment (Ward et al., 1989 Nature 341:544-546) consisting of a V_{H} domain; an isolated complementarity determining region (CDR); and an F(ab′)₂ fragment, a bivalent fragment comprising two Fab′ fragments linked by a disulfide bridge at the hinge region. These antibody fragments are obtained using conventional techniques well-known to those with skill in the art, and the fragments are screened for utility in the same manner as are intact antibodies. The term “antibody” is further intended to include bispecific and chimeric molecules having at least one antigen binding determinant derived from an antibody molecule.

In the diagnostic and prognostic assays of the invention, the antibody can be a polyclonal antibody or a monoclonal antibody and in a preferred embodiment is a labeled antibody.

In one embodiment, the invention provides a method for detecting the total amount of Pinl in a biological sample. In a related embodiment, the invention provides a method of detecting the amount of unphosphorylated Pinl in a biological sample. In yet another embodiment, the invention provides a method for detecting the amount of phosphorylated Pinl in a biological sample. The invention also provides a method of determining the amount of phosphorylated Pinl relative to the amount of unphosphorylated Pinl in a sample. Accordingly, the invention provides antibodies that recognize phosphorylated Pinl and unphospyorylated Pinl, antibodies that are specific for phosphorylated Pinl, and antibodies that are specific for unphosphorylated Pinl.

Polyclonal antibodies are produced by immunizing animals, usually a mammal, by multiple subcutaneous or intraperitoneal injections of an immunogen (antigen) and an adjuvant as appropriate. As an illustrative embodiment, animals are typically immunized against a protein, peptide or derivative by combining about 1 μg to 1 mg of protein capable of eliciting an immune response, along with an enhancing carrier preparation, such as Freund's complete adjuvant, or an aggregating agent such as alum, and injecting the composition intradermally at multiple sites. Animals are later boosted with at least one subsequent administration of a lower amount, as ⅕ to 1/10 the original amount of immunogen in Freund's complete adjuvant (or other suitable adjuvant) by subcutaneous injection at multiple sites. Animals are subsequently bled, serum assayed to determine the specific antibody titer, and the animals are again boosted and assayed until the titer of antibody no longer increases (i.e., plateaus).

Such populations of antibody molecules are referred to as “polyclonal” because the population comprises a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p. 246). Affinities range from low, e.g. 10⁻⁶ M, to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

“Monoclonal antibody” or “monoclonal antibody composition” as used herein refers to a preparation of antibody molecules of single molecular composition. A monoclonal antibody composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495-497; see also Brown et al. 1981 J. Immunol 127:539-46; Brown et al., 1980, J Biol Chem 255:4980-83; Yeh et al., 1976, PNAS 76:2927-31; and Yeh et al., 1982, Int. J. Cancer 29:269-75) and the more recent human B cell hybridoma technique (Kozbor et al., 1983, Immunol Today 4:72), EBV-hybridoma technique (Cole et al., 1985, Monoclonal Antibodies and Cancer Therapy, Alan R. Liss, Inc., pp. 77-96), and trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

“Labeled antibody” as used herein includes antibodies that are labeled by a detectable means and includes enzymatically, radioactively, fluorescently, chemiluminescently, and/or bioluminescently labeled antibodies.

One of the ways in which an antibody can be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the Pinl-specific or a cancer associated polypeptide-specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may be accomplished using any of a variety of immunoassays. For example, by radioactively labeling an antibody, it is possible to detect the antibody through the use of radioimmune assays. A description of a radioimmune assay (RIA) may be found in Laboratory Techniques and Biochemistry in Molecular Biology, by Work, T. S., et al., North Holland Publishing Company, NY (1978), with particular reference to the chapter entitled “An Introduction to Radioimmune Assay and Related Techniques” by Chard, T.

The radioactive isotope can be detected by such means as the use of a gamma counter or a scintillation counter or by audioradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In the diagnostic and prognostic assays of the invention, the amount of binding of the antibody to the biological sample can be determined by the intensity of the signal emitted by the labeled antibody and/or by the number cells in the biological sample bound to the labeled antibody.

Serum Assays

A serum assay for detecting a cancer marker is a non-evasive method, which is more acceptable to patients and also provides a tool for screening large number of samples. Additional advantages include that the antibody recognizes an antigen that is related to the early events rather than the later stages of progression to the metastatic phenotype. Serum assays can be used in conjunction with other assays presented herein to diagnose cancer.

Antibodies directed toward a protein of interest can be connected to magnetic beads and used to enrich a population. Immunomagnetic selection has been used previously for this purpose and examples of this method can be found, for example, at U.S. patent Ser. No. 5,646,001; Ree et al. (2002) Int. J. Cancer 97:28-33; Molnar et al. (2001) Clin. Cancer Research 7:4080-4085; and Kasimir-Bauer et al. (2001) Breast Cancer Res. Treat. 69:123-32. An antibody, either polyclonal or monoclonal, that is specific for a cell surface protein on a cell of interest is attached to a magnetic substrate thereby allowing selection of only those cells that express the surface protein of interest. Once a population of cells is selected, the following assays can be performed to test for the presence of Pinl.

Immunoassays

The amount of an antigen (i.e. Pinl or a cancer associated polypeptide) in a biological sample may be determined by a radioimmunoassay, an immunoradiometric assay, and/or an enzyme immunoassay.

“Radioimmunoassay” is a technique for detecting and measuring the concentration of an antigen using a labeled (i.e. radioactively labeled) form of the antigen. Examples of radioactive labels for antigens include ³H, ¹⁴C, and ¹²⁵I. The concentration of antigen in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to Pinl) is linked to a solid phase (i.e. a microtiter plate) and exposed to a biological sample containing antigen (i.e. Pinl). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for.

In a “competitive ELISA”, antibody is incubated with a sample containing antigen (i.e. Pinl). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

In a “immunohistochemistry assay” a section of tissue for is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or β-galactosidase), or chemical methods (e.g., DAB/Substrate chromagen).

B. Nucleic Acid-Based Diagnostic and Prognostic Methods

Also encompassed by this invention is a method of diagnosing cancer in a subject, comprising: detecting a level of Pinl and/or a cancer associated polypeptide nucleic acid in a biological sample; and comparing the level of Pinl and/or a cancer associated polypeptide in the biological sample with a level of Pinl in a control sample, wherein an elevation in the level of Pinl in the biological sample compared to the control sample is indicative cancer.

In addition, this invention pertains to a method of diagnosing cancer in a subject, comprising the steps of: detecting a level of Pinl or a cancer associated polypeptide nucleic acid in a biological sample; and comparing the level of Pinl and/or a cancer associated polypeptide in the biological sample with a level of Pinl and/or a cancer associated polypeptide in a control sample, wherein an elevation in the level of Pinl and/or a cancer associated polypeptide in the biological sample compared to the control sample is indicative of cancer.

In an embodiment of the above methods, the detecting a level of Pinl and/or a cancer associated polypeptide nucleic acid in a biological sample includes amplifying Pinl and/or a cancer associated polypeptide RNA. In another embodiment of the above methods, the detecting a level of Pinl and/or a cancer associated polypeptide nucleic acid in a biological sample includes hybridizing the Pinl and/or a cancer associated polypeptide RNA with a probe.

As an alternative to making determinations based on the absolute expression level of the Pinl and/or a cancer associated polypeptide, determinations may be based on the normalized expression level of Pinl and/or a cancer associated polypeptide. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-prostate cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the biological sample (absolute level of expression) is then divided by the mean expression value obtained for that marker. This provides a relative expression level.

One preferred diagnostic method for the detection of mRNA levels involves contacting the isolated mRNA with a nucleic acid molecule (probe) that can hybridize to the mRNA encoded by the gene being detected. Probes based on the sequence of a nucleic acid molecule of the invention can be used to detect transcripts corresponding to Pin-1. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Hybridization of an mRNA with the probe indicates that the marker in question is being expressed. In an embodiment, the probe includes a label group attached thereto, e.g., a radioisotope, a fluorescent compound, an enzyme, or an enzyme co-factor.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

“Amplifying” refers to template-dependent processes and vector-mediated propagation which result in an increase in the concentration of a specific nucleic acid molecule relative to its initial concentration, or in an increase in the concentration of a detectable signal. As used herein, the term template-dependent process is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, J. D. et al., In: Molecular Biology of the Gene, 4th Ed., W. A. Benjamin, Inc., Menlo Park, Calif. (1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by Cohen et al. (U.S. Pat. No. 4,237,224), Maniatis, T. et al., Molecular Cloning (A Laboratory Manual), Cold Spring Harbor Laboratory, 1982.

A number of template dependent processes are available to amplify the target sequences of interest present in a sample. One of the best known amplification methods is the polymerase chain reaction (PCR) which is described in detail in Mullis, et al., U.S. Pat. No. 4,683,195, Mullis, et al., U.S. Pat. No. 4,683,202, and Mullis, et al., U.S. Pat. No. 4,800,159, and in Innis et al., PCR Protocols, Academic Press, Inc., San Diego Calif., 1990. Briefly, in PCR, two primer sequences are prepared which are complementary to regions on opposite complementary strands of the target sequence. An excess of deoxynucleoside triphosphates are added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. Preferably a reverse transcriptase PCR amplification procedure may be performed in order to quantify the amount of mRNA amplified. Polymerase chain reaction methodologies are well known in the art.

Another method for amplification is the ligase chain reaction (LCR), disclosed in European Patent No. 320,308B1. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. Whiteley, et al., U.S. Pat. No. 4,883,750 describes an alternative method of amplification similar to LCR for binding probe pairs to a target sequence.

Qbeta Replicase, described in PCT Application No. PCT/US87/00880 may also be used as still another amplification method in the present invention. In this method, a replicative sequence of RNA which has a region complementary to that of a target is added to a sample in the presence of an RNA polymerase. The polymerase will copy the replicative sequence which can then be detected.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Pinl and/or a cancer associated polypeptides can also be detected using a cyclic probe reaction (CPR). In CPR, a probe having a 3′ and 5′ sequences of non-prostate specific DNA and middle sequence of prostate specific RNA is hybridized to DNA which is present in a sample. Upon hybridization, the reaction is treated with RNaseH, and the products of the probe identified as distinctive products generating a signal which are released after digestion. The original template is annealed to another cycling probe and the reaction is repeated. Thus, CPR involves amplifying a signal generated by hybridization of a probe to a prostate cancer specific expressed nucleic acid.

Still other amplification methods described in GB Application No. 2 202 328, and in PCT Application No. PCT/US89/01025 may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh D., et al., Proc. Natl. Acad. Sci. (U.S.A.) 1989, 86:1173, Gingeras T. R., et al., PCT Application WO 88/1D315), including nucleic acid sequence based amplification (NASBA) and 3SR. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has prostate specific sequences. Following polymerization, DNA/RNA hybrids are digested with RNase H while double stranded DNA molecules are heat denatured again. In either case the single stranded DNA is made fully double stranded by addition of second prostate specific primer, followed by polymerization. The double stranded DNA molecules are then multiply transcribed by a polymerase such as T7 or SP6. In an isothermal cyclic reaction, the RNAs are reverse transcribed into double stranded DNA, and transcribed once against with a polymerase such as T7 or SP6. The resulting products, whether truncated or complete, indicate prostate cancer specific sequences.

Davey, C., et al., European Patent No. 329,822B1 disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller, H. I., et al., PCT Application WO 89/06700 discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “race” disclosed by Frohman, M. A., In: PCR Protocols: A Guide to Methods and Applications 1990, Academic Press, New York) and “one-sided PCR” (Ohara, O., et al., Proc. Natl. Acad. Sci. (U.S.A.) 1989, 86:5673-5677).

Methods based on ligation of two (or more) oligonucleotides in the presence of nucleic acid having the sequence of the resulting “di-oligonucleotide”, thereby amplifying the di-oligonucleotide (Wu, D. Y. et al., Genomics 1989, 4:560), may also be used in the amplification step of the present invention.

Following amplification, the presence or absence of the amplification product may be detected. The amplified product may be sequenced by any method known in the art, including and not limited to the Maxam and Gilbert method. The sequenced amplified product is then compared to a sequence known to be in a prostate cancer specific sequence. Alternatively, the nucleic acids may be fragmented into varying sizes of discrete fragments. For example, DNA fragments may be separated according to molecular weight by methods such as and not limited to electrophoresis through an agarose gel matrix. The gels are then analyzed by Southern hybridization. Briefly, DNA in the gel is transferred to a hybridization substrate or matrix such as and not limited to a nitrocellulose sheet and a nylon membrane. A labeled probe is applied to the matrix under selected hybridization conditions so as to hybridize with complementary DNA localized on the matrix. The probe may be of a length capable of forming a stable duplex. The probe may have a size range of about 200 to about 10,000 nucleotides in length, preferably about 200 nucleotides in length. Various labels for visualization or detection are known to those of skill in the art, such as and not limited to fluorescent staining, ethidium bromide staining for example, avidin/biotin, radioactive labeling such as ³²P labeling, and the like. Preferably, the product, such as the PCR product, may be run on an agarose gel and visualized using a stain such as ethidium bromide. The matrix may then be analyzed by autoradiography to locate particular fragments which hybridize to the probe.

C. Assays for Use in Combination with Pinl Detection

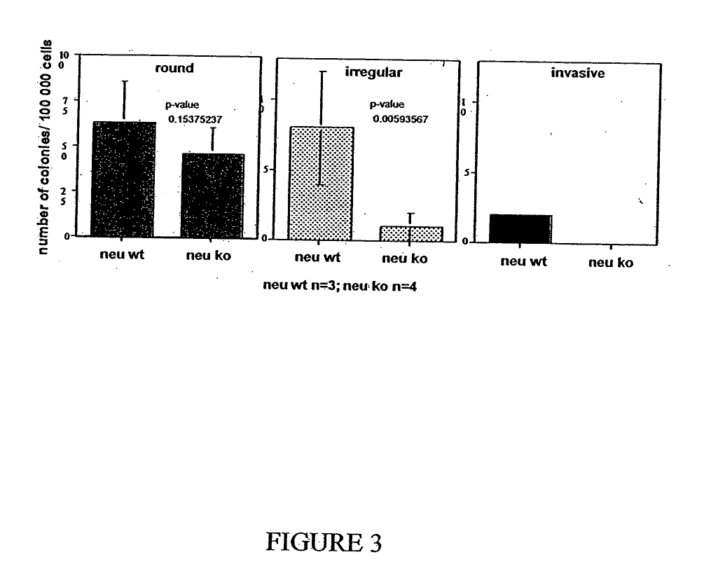

In one embodiment, the invention provides a method of determining if the invasive potential of a primary cell. In certain embodiments the cell is a primary epithelial cell isolated from a subject. In a specific embodiment, the primary cell is an epithelial cell isolated from the breast tissue. The method provided allows for the isolation and growth of a primary cell on a matrix to see the morphology that the cell colonies develop. Invasive growth of the colonies indicate that the cells will develop into infiltrating carcinomas in a subject. This method allows for a physician to determine the aggressiveness potential of a premalignant lesion and to adjust a subject's therapy accordingly. Example 2 and FIG. 3 describe this method further.

III. Therapeutic Methods

Once it has been determined that a subject will benefit from treatment using a Pinl inhibitor or a Pinl inhibitor in combination with a cancer associated polypeptide inhibitor using the diagnostic methods described herein, the subject can be treated using the methods described below.

A subject that expresses a cancer associated polypeptide, e.g., the polypeptide encoded by her2/neu, can be administered a Pinl inhibitor. Additionally, a second anticancer treatment may be administered to the subject. The second anticancer treatment can be, for example, a cancer associated polypeptide inhibitor, or a compound that alters the expression of a cancer associated polypeptide. In another embodiment, the second anticancer treatment can be radiation. In one specific embodiment the second anticancer composition is herceptin. Other cancer compositions that can be used with the methods of the invention are (Adriamycin) Doxorubicin, Aldesleukin or IL-2 (Proleukin), Amsacrine (acridinyl anisidide; m-AMSA), Asparaginase, Bleomycin, Busulphan, (Campto) Irinotecan, Capecitabine (Xeloda), Carboplatin (Paraplatin, JM8),Carmustine (BCNU), Chlorambucil, Cisplatin, Cladribine (2-CdA, Leustatin), Cyclophosphamide, Cytarabine (Ara C, cytosine arabinoside), Dacarbazine (DTIC), Dactinomycin (Actinomycin D), Daunorubicin, Docetaxel (Taxotere), Doxorubicin (Adriamycin), Epirubicin, Estramustine (Emcyt, Estracyte), Etoposide (VP16, Etopophos), Fludarabine, Fluorouracil (5FU), Gemcitabine (Gemzar), (Herceptin) Trastuzumab, Hydroxyurea, Idarubicin, (Zavedos), Ifosfamide, Interferon (Roferon, Intron A), Irinotecan (Campto), Lomustine (CCNU), Melphalan, Mercaptopurine (6-MP, Purinethol), Methotrexate, Mitomycin C, Mitozantrone, Mustine (Chlormethine), Oxaliplatin, Paclitaxel (Taxol), Pentostatin, Procarbazine, Raltitrexed (Tomudex), Streptozocin (Zanosar), (Taxol) Paclitaxel, (Taxotere) Docetaxel, Tegafur with uracil (Uftoral), Temozolomide (Temodal), Thioguanine (Lanvis, 6-TG, 6-thioguanine, Tabloid), Thiotepa (Thioplex, Triethylenethiophosphoramide), (Tomudex) Raltitrexed, Topotecan (Hycamtin), Trastuzumab (Herceptin), Tretinoin (Vesanoid), Vinblastine (Velban), Vincristine (Oncovin), Vindesine (Eldisine), Vinorelbine (Navelbine)

In a particular embodiment, the Pinl inhibitor can be administered to a subject that has already received an initial anticancer treatment with, for example, one of the above indicated cancer therapeutics. In one embodiment, the subject is resistant to the initial treatment and is administered a Pinl inhibitor subsequent to developing resistance. As used herein, “resistant” includes subjects that are naturally resistant to a given treatment, or subjects that have developed resistance after having been treated with a given compound.

In another specific embodiment, a subject is administered a Pinl inhibitor in combination with a second anticancer treatment specific for a cancer associated polypeptide. In a related embodiment, a subject is administered an amount of each inhibitor that is different than the amount of each required if the two inhibitors were administered alone. In one example, the amount of one or more of the inhibitors is less than the amount required if administered alone. This proves advantageous when a given anticancer treatment is toxic to a patient and reducing the amount administered would benefit the subject.

IV. Screening Assays

In order to determine if a compounds described herein has the ability to modulate the expression or activity of Pinl or a cancer associated polypeptide, the following screening assays can by used.

The invention provides a method (also referred to herein as a “screening assay”) for testing candidate compounds or agents (as described above) which ameliorate, prevent or delay one or more neurodegenerative phenotypes associated with a neurodegenerative disorder.

The invention provides in vivo and in vitro methods of identifying agents that are capable of being used in the methods of the invention.

A. In Vitro Methods

In certain embodiments, the candidate compounds are first examined in vitro in a cell-based assay comprising contacting a cell expressing PINl with a test compound and determining the ability of the test compound to modulate (e.g., stimulate) the activity of the PINl target molecule. Cell based assays useful for examining Pinl activity are well-known in the art, and can found, for example, U.S. Pat. Nos. 6,258,582, 6,462,173B1, 6,495,376, U.S. patent application US2002/025521, and Fisher et al. (Biomed. Biochim. Acta, 1984, 43: 1101-1111), the entire contents each of which are expressly incorporated herein by reference.

In further embodiments, the ability of a compound to modulate Pinl protein degradation, or to decrease Pinl phosphorylation can be tested using methods described, for example, in Basu, et al. 2002) Neoplasia 4, 218-227, and Lu, et al., J. Biol. Chem. 277:2381-2384.

A further in vitro method is a three dimensional plate assay as described in Example 2 wherein a compounds ability to prevent a cell from developing a invasive phenotype characteristic of invasive cancer.

B. In Vivo Methods

The animal model described herein can be used to further test the candidate compounds identified using the in vitro methods of the invention. Transgenic PINl misexpressing animals that express a cancer associated polypeptide, e.g., mice, or cells can be used to screen for treatments. The candidate treatment can be administered over a range of doses to the animal or cell. Efficacy can be assayed at various time points for the effects of the compound on the treatment or prevention of the disorder being evaluated. For example, use of compounds for the treatment or prevention of cancer includes treatment of the animal to thereby identify treatments suitable for administration to human subjects. Such treatments can be evaluated by determining the effect of the treatment on the onset, progression or reversal of cancer.

V. Inhibitory Compounds

Varieties of inhibitory compounds are known in the art and can be employed in the methods of the invention. Suitable compounds include those that decrease the biological activity of Pinl and cancer associated polypeptides including, but not limited to, those that increase or increase the rate of Pinl and cancer associated polypeptides degradation, modulate Pinl phosphorylation, decrease Pinl catalytic activity, decrease the activity of a cancer associated polypeptide and/or decrease Pinl or cancer associated polypeptide expression (e.g., by gene therapy). Such compounds can be identified by a number of art recognized assays such as those described herein.

For example, agents that decrease the biological activity of Pinl or cancer associated polypeptides can be derived using Pinl or cancer associated polypeptides nucleic acid or amino acid sequences. The nucleotide and amino acid sequences of these molecules are known in the art and can be found in the literature or on a database such as GenBank. See, for example, Pinl (Lu, K. P. et al. (1996) Nature. 380544-7 or GenBank Accession number AAC50492 or U49070).

A. Nucleic Acid Molecules

Nucleic acid molecules can also be used as modulators of Pinl or cancer associated polypeptides activity or expression.

Given the sequences encoding Pinl and cancer associated polypeptides disclosed in the art, a nucleic acid for use in the methods of the invention can be constructed using chemical synthesis and enzymatic ligation reactions using procedures known in the art. For example, a nucleic acid molecule can be chemically or recombinantly synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

In yet another embodiment, the Pinl or cancer associated polypeptide nucleic acid molecules of the present invention can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

Nucleic acid molecules of the invention can be produced by inserting the nucleic acid molecule into a vector and producing multiple copies of the vector and then isolating the nucleic acid sequence that encodes Pinl, a portion of Pinl, a cancer associated polypeptide, or a fragment of a cancer associated polypeptide.

B. Proteins and Peptides

In addition to the full length polypeptides, a number of useful peptides can also be derived from Pinl and cancer associated polypeptide sequences. A peptide may, for instance, be fragment of the naturally occurring protein, or a mimic or peptidomimetic. Variants of Pinl or cancer associated polypeptides which can be generated by mutagenesis (e.g., amino acid substitution, amino acid insertion, or truncation), and identified by screening combinatorial libraries of mutants, such as truncation mutants, of a protein for the desired activity.

For example, a variegated library of Pinl or cancer associated polypeptides variants can be generated by combinatorial mutagenesis at the nucleic acid level, for example, by enzymatically ligating a mixture of synthetic oligonucleotides into gene sequences such that a degenerate set of potential Pinl sequences is expressible as individual polypeptides, or alternatively, as a set of larger fusion proteins (e.g., for phage display) containing the set of sequences therein. Chemical synthesis of a degenerate gene sequence can also be performed in an automatic DNA synthesizer, and the synthetic gene then ligated into an appropriate expression vector. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

Once suitable polypeptides are identified, systematic substitution of one or more amino acids of the amino acid sequence, or a functional variant thereof, with a D-amino acid of the same type (e.g., D-lysine in place of L-lysine) can also be used to generate a peptide which has increased stability. In addition, constrained peptides comprising a polypeptide sequence, a functional variant thereof, or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

Peptides can be produced recombinantly or direct chemical synthesis. Further, peptides may be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, and desirable pharmacokinetic properties.

The invention further provides a peptide analog or peptide mimetic of the Pinl or cancer associated polypeptides. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to Pinl or functional variants thereof can be used to produce an antagonistic effect. Generally, peptidomimetics are structurally similar to the paradigm polypeptide (Pinl) but have one or more peptide linkages optionally replaced by a linkage selected from the group consisting of: —CH2NH—, —CH2S—, —CH2-CH2—, —CH═CH— (cis and trans), —COCH2-, —CH(OH)CH2-, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference.

C. Small Molecules

Small molecules of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

Small molecule inhibitors of Pinl are described in U.S. Provisional Application No. 60/488262, filed Jul. 18, 2003, entitled PIN-1 MODULATING COMPOUNDS AND METHODS OF USE THEREOF; U.S. Provisional application Ser. No. 10/379,115, filed Mar. 3, 2003, entitled “Methods for Designing Specific Inhibitors for PINl Proline Isomerase and PINl-Related Molecules”; U.S. Provisional Application No. 60/361,206 filed Mar. 1, 2002, entitled “Pinl-Modulating Compounds and Methods of Use Thereof”; U.S. Provisional Application Ser. No. 60/361,246, filed Mar. 1, 2002, entitled “Pinl-Modulating Compounds and Methods of Use Thereof”; U.S. Provisional Application Ser. No. 60/361,231, filed Mar. 1, 2002, entitled “Pinl-Modulating Compounds and Methods of Use Thereof”; U.S. Provisional Application Ser. No. 60/361,227, filed on Mar. 1, 2002; entitled “Methods for Designing Specific Inhibitors for Pinl Proline Isomerase and Pinl-Related Molecules”; U.S. Provisional Application No. 60/360,799 filed Mar. 1, 2002, entitled “Methods of Treating Pinl Associated Disorders”; U.S. Provisional Application No. 60/451,807, entitled “Pinl -Modulating Compounds and Methods of Use Thereof“, filed Mar. 3, 2003; U.S. Provisional Application No. 60/463271, entitled “Photochemotherapeutic Compounds for Use in Treatment of Pinl Associated States”, filed Apr. 16, 2003; and U.S. Provisional Application No. 60/451,838, entitled “Pinl-Modulating Compounds and Methods of Use Thereof”, filed Mar. 3, 2003, the contents of which are expressly incorporated herein by reference.

D. Antibodies

In another embodiment, the invention employs antibodies to inactivate Pinl and/or a cancer associated polypeptide. As used herein, the term “antibody” includes whole antibodies or antigen-binding fragments thereof including, for example, Fab, F(ab′)2, Fv and single chain Fv fragments. Suitable antibodies include any form of antibody, e.g., murine, human, chimeric, or humanized and any type antibody isotype, such as IgG1, IgG2, IgG3, IgG4, IgM, IgA1, IgA2, IgAsec, IgD, or IgE isotypes.

Antibodies which specifically bind Pinl or cancer associated polypeptides can serve as an antagonists of Pinl or the cancer associated polypeptide. As used herein, “specific binding” refers to antibody binding to a predetermined antigen. Typically, the antibody binds with a dissociation constant (KD) of 10−7 M or less, and binds to the predetermined antigen with a KD that is at least two-fold less than its KD for binding to a non-specific antigen (e.g., BSA, casein) other than the predetermined antigen or a closely-related antigen. Several Pinl antibodies are known, (see, for example, U.S. Pat. 6,596,848).

Alternatively, antibodies can be produced according to well known methods for antibody production, and tested for agonist activity using the methods described herein. For example, antigenic peptides of Pinl which are useful for the generation of antibodies can be identified in a variety of manners well known in the art. For example, useful epitopes can be predicted by analyzing the sequence of the protein using web-based predictive algorithms (BIMAS & SYFPEITHI) to generate potential antigenic peptides from which synthetic versions can be made and tested for their capacity to generate Pinl specific antibodies.

The antibodies can be monoclonal or polyclonal. The terms “monoclonal antibodies” as used herein, refers to a population of antibody molecules that contain only one species of an antigen binding site capable of immunoreacting with a particular epitope of an antigen, whereas the term “polyclonal antibodies” refers to a population of antibody molecules that contain multiple species of antigen binding sites capable of interacting with a particular antigen. Techniques for generating monoclonal and polyclonal antibodies are well known in the art (See, e.g., Current Protocols in Immunology, Coligan et al., eds., John Wiley & Sons, http://www.does.org/masterli/cpi.html).

Recombinant antibodies, such as chimeric and humanized monoclonal antibodies, comprising both human and non-human portions can be made using standard recombinant DNA techniques, and are also within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; U.S. Pat. Nos. 5,225,539 5,565,332, 5,871,907, or 5,733,743; Jones et al. (1986) Nature 321:552-525; Verhoeyan et al. (1988) Science 239:1534; and Beidler et al. (1988) J. Immunol. 141:4053-4060.

Recombinant chimeric antibodies can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

Fully human antibodies that bind Pinl or cancer associated polypeptides can also be employed in the invention, and can be produced using techniques that are known in the art. For example, transgenic mice can be made using standard methods, e.g., according to Hogan, et al., “Manipulating the Mouse Embryo: A Laboratory Manual”, Cold Spring Harbor Laboratory, which is incorporated herein by reference, or are purchased commercially. Embryonic stem cells are manipulated according to published procedures (Teratocarcinomas and embryonic stem cells: a practical approach, Robertson, E. J. ed., IRL Press, Washington, D.C., 1987; Zjilstra et al. (1989) Nature 342:435-438; and Schwartzberg et al. (1989) Science 246:799-803, each of which is incorporated herein by reference). For example, transgenic mice can be immunized using purified or recombinant Pinl or a fusion protein comprising at least an immunogenic portion of Pinl. Antibody reactivity can be measured using standard methods. The term “recombinant human antibody,” as used herein, includes all human antibodies that are prepared, expressed, created or isolated by recombinant means. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Single chain antagonistic antibodies that bind to Pinl, a cancer associated polypeptides, or their respective ligand or receptor also can be identified and isolated by screening a combinatorial library of human immunoglobulin sequences displayed on M13 bacteriophage ( Winter et al. 1994 Annu. Rev. Immunol. 1994 12:433; Hoogenboom et al., 1998, Immunotechnology 4: 1).

In yet another embodiment of the invention, bispecific or multispecific antibodies that bind to Pinl, a cancer associated polypeptide, or antigen-binding portions thereof. Such antibodies can be generated, for example, by linking one antibody or antigen-binding portion (e.g., by chemical coupling, genetic fusion, noncovalent association or otherwise) to a second antibody or antigen-binding portion. Bispecific and multispecific molecules of the present invention can be made using chemical techniques, “polydoma” techniques or recombinant DNA techniques. Bispecific and multispecific molecules can also be single chain molecules or may comprise at least two single chain molecules. Methods for preparing bi- and multispecific molecules are described for example in D. M. Kranz et al. (1981) Proc. Natl. Acad. Sci. USA 78:5807; U.S. Pat. No. 4,474,893; U.S. Pat. No. 5,260,203; U.S. Pat. No. 5,534,254. U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcR and triggers at least one effector function.

VI. Pharmaceutical Compositions

The Pinl inhibitors and cancer associated polypeptide inhibitors (also referred to herein as “active compounds”) of the invention can be incorporated into pharmaceutical compositions suitable for administration. The compositions can be individual compositions each of which contains an inhibitor and a pharmaceutically acceptable carrier, e.g., a Pinl inhibitor and a pharmaceutically acceptable carrier, or a composition that contains more than one inhibitor and a pharmaceutically carrier, e.g., a Pinl inhibitor and a second cancer associated polypeptide inhibitor. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

VII. Animals

The invention provides a transgenic animals. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous Pinl gene has been altered by homologous recombination between the endogenous gene and an exogenous DNA molecule introduced into a cell of the animal, e.g., an embryonic cell of the animal, prior to development of the animal. In specific embodiments the animal of the invention is a Pinl misexpressing mouse (for example, as described in Fujimori, et al. (1999) Biochem. Biophys. Res. Commun. 265:658-63) that expresses a cancer associated polypeptide transgene. In a specific embodiment, the cancer associated polypeptide transgene is MMTV-Ras or MMTV-Her2/nue. The transgenic mouse of the invention can be used to determine if effects of the expression of the transgene, e.g., the development of cancer, can be overcome by a Pinl inhibitor.

In preferred embodiments, misexpression of the gene encoding the PINl protein is caused by disruption of the PINl gene. For example, the PINl gene can be disrupted through removal of DNA encoding all or part of the protein.

In preferred embodiments, the animal can be heterozygous or homozygous for a misexpressed PINl gene, e.g., it can be a transgenic animal heterozygous or homozygous for a PINl transgene.

In preferred embodiments, the animal is a transgenic mouse with a transgenic disruption of the PINl gene, preferably an insertion or deletion, which inactivates the gene product. The nucleotide sequence of the wild type PINl is known in the art and described in, for example, U.S Pat. No. 5,972,697, the contents of which are incorporated herein by reference. Preferred embodiments also include animals in which one or more genes, in addition to Pinl, are misexpressed.

In another preferred embodiment, the animal is a transgenic animal that expresses Pinl and a cancer associated polypeptide. In certain embodiments the animal expresses Pinl and an oncogene.

A. Generation of Transgenic Mice

The invention provides methods of making mice that express a cancer associated polypeptide transgene and/or misexpresses Pinl.

B. Knock-out Construct

The nucleotide sequence to be used in producing the targeting construct is digested with a particular restriction enzyme selected to digest at a location(s) such that a new DNA sequence encoding a marker gene can be inserted in the proper position within this nucleotide sequence. The marker gene should be inserted such that it can serve to prevent expression of the native gene. The position will depend on various factors such as the restriction sites in the sequence to be cut, and whether an exon sequence or a promoter sequence, or both is (are) to be interrupted (i.e., the precise location of insertion necessary to inhibit gene expression). In some cases, it will be desirable to actually remove a portion or even all of one or more exons of the gene to be suppressed so as to keep the length of the targeting construct comparable to the original genomic sequence when the marker gene is inserted in the targeting construct. In these cases, the genomic DNA is cut with appropriate restriction endonucleases such that a fragment of the proper size can be removed.

The marker sequence can be any nucleotide sequence that is detectable and/or assayable. For example, the marker gene can be an antibiotic resistance gene or other gene whose expression in the genome can easily be detected. The marker gene can be linked to its own promoter or to another strong promoter from any source that will be active in the cell into which it is inserted; or it can be transcribed using the promoter of the PINl gene. The marker gene can also have a polyA sequence attached to the 3′ end of the gene; this sequence serves to terminate transcription of the gene. For example, the marker sequence can be a protein that (a) confers resistance to antibiotics or other toxins; e.g., ampicillin, tetracycline, or kanamycin for prokaryotic host cells, and neomycin, hygromycin, or methotrexate for mammalian cells; (b) complements auxotrophic deficiencies of the cell; or (c) supplies critical nutrients not available from complex media.

After the DNA sequence has been digested with the appropriate restriction enzymes, the marker gene sequence is ligated into the PINl DNA sequence using methods known to the skilled artisan and described in Sambrook et al., Molecular Cloning A Laboratory Manual, 2nd Ed., ed., Cold Spring Harbor Laboratory Press: 1989, the contents of which are incorporated herein by reference.

Preferably, the ends of the DNA fragments to be ligated are compatible; this is accomplished by either restricting all fragments with enzymes that generate compatible ends, or by blunting the ends prior to ligation. Blunting is performed using methods known in the art, such as for example by the use of Klenow fragment (DNA polymerase I) to fill in sticky ends.

The ligated targeting construct can be inserted directly into embryonic stem cells, or it may first be placed into a suitable vector for amplification prior to insertion. Preferred vectors are those that are rapidly amplified in bacterial cells such as the pBluescript II SK vector (Stratagene, San Diego, Calif.) or pGEM7 (Promega Corp., Madison, Wisc).

C. Construct for Conditional Expression of Pinl

Conditional neuron-specific deletion of Pinl can be generated using Cre- and loxP-mediated recombination using standard techniques. As the first step to reach this goal, mouse genomic BAC clones covering the Pinl gene can be obtained from Incite Genetics. To generate the targeting vector, three Pinl genomic fragments will be subcloned into the pflox vector, which consists of a selection marker PGK-Neo cassette flanked by two loxP sites and a third loxP site.

D. Transfection of Embryonic Stem Cells

Mouse embryonic stem cells (ES cells) can be used to generate the transgenic mice. Any ES cell line that is capable of integrating into and becoming part of the germ line of a developing embryo, so as to create germ line transmission of the targeting construct is suitable for use herein. For example, a mouse strain that can be used for production of ES cells is the 129J strain. A preferred ES cell line is murine cell line D3 (American Type Culture Collection catalog no. CRL 1934). The cells can be cultured and prepared for DNA insertion using methods known in the art and described in Robertson, Teratocarcinomas and Embryonic Stem Cells: A Practical Approach, E. J. Robertson, ed. IRL Press, Washington, D.C., 1987, in Bradley et al., Current Topics in Devel. Biol., 20:357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The knockout construct can be introduced into the ES cells by methods known in the art, e.g., those described in Sambrook et al. Suitable methods include electroporation, microinjection, and calcium phosphate treatment methods.

The targeting construct to be introduced into the ES cell is preferably linear. Linearization can be accomplished by digesting the DNA with a suitable restriction endonuclease selected to cut only within the vector sequence and not within the gene sequence.

After the introduction of the targeting construct, the cells are screened for the presence of the construct. The cells can be screened using a variety of methods. Where the marker gene is an antibiotic resistance gene, the cells can be cultured in the presence of an otherwise lethal concentration of antibiotic. Those cells that survive have presumably integrated the knockout construct. A southern blot of the ES cell genomic DNA can also be used. If the marker gene is a gene that encodes an enzyme whose activity can be detected (e.g., beta-galactosidase), the enzyme substrate can be added to the cells under suitable conditions, and the enzymatic activity can be analyzed.

To identify those cells with proper integration of the targeting construct, the DNA can be extracted from the ES cells using standard methods. The DNA can then be probed on a southern blot with a probe or probes designed to hybridize in a specific pattern to genomic DNA digested with particular restriction enzymes. Alternatively, or additionally, the genomic DNA can be amplified by PCR with probes specifically designed to amplify DNA fragments of a particular size and sequence such that, only those cells containing the targeting construct in the proper position will generate DNA fragments of the proper size.

E. Injection/Implantation of Embryos

Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals. In an exemplary embodiment, mouse zygotes are collected from six-week old females that have been super ovulated with pregnant mares serum (PMS) followed 48 hours later with human chorionic gonadotropin. Primed females are placed with males and checked for vaginal plugs on the following morning. Pseudo pregnant females are selected for estrus, placed with proven sterile vasectomized males and used as recipients. Zygotes are collected and cumulus cells removed. Furthermore, blastocytes can be harvested. Pronuclear embryos are recovered from female mice mated to males. Females are treated with pregnant mare serum, PMS, to induce follicular growth and human chorionic gonadotropin, hCG, to induce ovulation. Embryos are recovered in a Dulbecco's modified phosphate buffered saline (DPBS) and maintained in Dulbecco's modified essential medium (DMEM) supplemented with 10% fetal bovine serum.

Microinjection of a targeting construct can be performed using standard micromanipulators attached to a microscope. For instance, embryos are typically held in 100 microliter drops of DPBS under oil while being microinjected. DNA solution is microinjected into the male pronucleus. Successful injection is monitored by swelling of the pronucleus. Recombinant ES cells can be injected into blastocytes, using similar techniques. Immediately after injection embryos are transferred to recipient females, e.g. mature mice mated to vasectomized male mice. In a general protocol, recipient females are anesthetized, paralumbar incisions are made to expose the oviducts, and the embryos are transformed into the ampullary region of the oviducts. The body wall is sutured and the skin closed with wound clips.

F. Screening for the Presence of the Targeting Construct

Transgenic animals can be identified after birth by standard protocols. DNA from tail tissue can be screened for the presence of the targeting construct using southern blots and/or PCR. Offspring that appear to be mosaics are then crossed to each other if they are believed to carry the targeting construct in their germ line to generate homozygous knockout animals. If it is unclear whether the offspring will have germ line transmission, they can be crossed with a parental or other strain and the offspring screened for heterozygosity. The heterozygotes are identified by southern blots and/or PCR amplification of the DNA.

The heterozygotes can then be crossed with each other to generate homozygous transgenic offspring. Homozygotes may be identified by southern blotting of equivalent amounts of genomic DNA from mice that are the product of this cross, as well as mice that are known heterozygotes and wild type mice. Probes to screen the southern blots can be designed as set forth above.

Other means of identifying and characterizing the knockout offspring are known in the art. For example, northern blots can be used to probe the mRNA for the presence or absence of transcripts encoding the gene knocked out, the marker gene, or both. In addition, western blots can be used to assess the level of expression of the gene knocked out in various tissues of these offspring by probing the western blot with an antibody against the protein encoded by the gene knocked out (e.g., the PINl protein), or an antibody against the marker gene product, where this gene is expressed. Finally, in situ analysis (such as fixing the cells and labeling with antibody) and/or FACS (fluorescence activated cell sorting) analysis of various cells from the offspring can be performed using suitable antibodies to look for the presence or absence of the targeting construct gene product.

G. Mice Containing Multiple Mutations

Transgenic mice containing mutations as described herein can be crossed with mice containing mutations in additional genes associated with cancer. Mice that are heterozygous or homozygous for each of the mutations can be generated and maintained using standard crossbreeding procedures. Examples of mice that can be bred with mice containing mutations, e.g., Pinl mutations, include those that overexpress a cancer associated polypeptide, e.g., an oncogene.

H. Other Transgenic Animals

The transgenic animal used in the methods of the invention can be a mammal; a bird; a reptile or an amphibian. Suitable mammals for uses described herein include: ruminants; ungulates; domesticated mammals; and dairy animals. Preferred animals include: goats, sheep, camels, cows, pigs, horses, oxen, llamas, chickens, geese, and turkeys. Methods for the preparation and use of such animals are known in the art. A protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic sheep can be found in Transgenic Animal Technology, A Handbook, 1994, ed., Carl A. Pinkert, Academic Press, Inc.

I. Use of Animals of the Invention

Animals of the invention can be used for determining if a subject that expresses a cancer associated polypeptide would be a candidate for treatment with a Pinl inhibitor. In one embodiment, a knock out Pinl animal that overexpresses a cancer associated polypeptide is tested for the development of cancer. If cancer development is delayed or does not occur, the subject that expresses the cancer associated polypeptide would likely benefit from treatment with a Pinl inhibitor. In a related embodiment, if the animal does not develop cancer, a transgenic animal that expresses Pinl and the cancer associated polypeptide can be used to screen for compounds, or combinations of compounds that would be useful in the treatment of cancer. In particular embodiments the compounds can be specific for Pinl or the cancer associated polypeptide.

The contents of all references, pending patent applications and published patents, cited throughout this application are hereby expressly incorporated herein by reference

### EXAMPLES

Experimental Procedures

The following procedures were used in one or more of the Examples described below.

Animals

MMTV-v-Ha-Ras, MMTV-c-myc (Sinn et al., 1987) or MMTV-c-Neu (Bouchard et al, 1989; Muller et al., 1988) transgenic mice in FVB genetic background were purchased from Charles River Laboratories. Transgenic animals were bred with Pinl−/− mice, which are in mixed genetic background of 129:C57BL6, as described (Liou et al., 2002). Transgenic heterozygous animals were then bred with heterozygous females to obtain the experimental cohort that was followed for the development of tumors. Only virgin females were enrolled in the study and they were examined for the development of tumors twice weekly. For histological sections, the glands were fixed in Bouin's solution, and standard histology sections were stained with Hematoxylin/Eosin. The slides were reviewed with a rodent histopathologist. For whole mount preparations, an inguinal gland was removed and stained with carmine red as described (Liou et al, 2002). Primary ducts, side branches and end buds were counted under a dissecting microscope. Immunohistochemistry to detect cyclin Dl, Ha-Ras and c-Neu was done as described (Liou et al., 2002).

Immunoblotting and Immunohistochemistry

Immunoblotting and immunohistochemistry were performed as described (Wulf et al., 2001). Briefly, tissue lysates from inguinal mammary glands were prepared and spun, followed by incubation for 10 min at 4° C. to allow for solidification of the fat component. The lower, liquid phase was aspirated. Immunoprecipitation experiments were done using antibody-coupled agarose beads for the c-Neu antigen (sc-7301 AC) and the H-ras antigen (sc-35 AC), while immunoblotting was done with antibodies sc-520 for H-Ras, and anti c-Neu Ab-3 from Oncogene. Polyclonal antibody sc 718 was used for immunoprecipitation and immunoblotting of cyclin Dl (sc 718), all antibodies except for anti c-Neu were purchased from Santa Cruz Biotechnology. For immunohistochemistry, both tissue sections and matrigel-embedded cultures were fixed with Bouin's solution and paraffin-embedded. The sections were deparaffinized, rehydrated and subjected to antigen retrieval by boiling them for 10 min in 1× Antigen retrieval solution (Vectra). Slides were blocked with PBS/5% goat serum, and then incubated with antibodies against Ha-Ras, cyclin Dl and c-Neu. They were then processed with biotinylated secondary antibody, and developed using the Vectorstain kit and DAB solution (Vector Labs).

Culture of Primary Mouse MECs ex vivo

Primary MECs were isolated from the morphologically and histologically normal mammary glands from virgin mice ages 3-4 months. The mammary glands were mechanically disaggregated, and then subjected to collagenase digestion (100 mg/ml) at 37° C. with gentle shaking (100 rpm) in a total volume of 10 ml DMEM/F12 per mammary gland for 2 hours. The digested material was then washed with HBSS/2% horse serum (Gibco) 3 times, followed by 1 wash with HBSS. The pellet was resuspended in trypsin and digested for another 10 min at 37° C., followed by neutralization with 10% horse serum, and a final wash with HBSS. The pellet was resuspended in MEGM and plated on 6 cm culture dishes that had been coated with collagen (50 mcg/ml). After 3-5 days in culture the mammary epithelial cells were trypsinized, washed with HBSS/10% horse serum, counted and resuspended in DMEM/F12 supplemented with Insulin 5 ng/ml, Choleratoxin 100 ng/ml, Hydrocortisone 500 ng/ml at 100,000 cells/ml. The suspension was then diluted 1:1 with MEGM/4% Matrigel (BD Biosciences 354230) and plated in Falcon Culture slides (BD 354118) that had been coated with Matrigel, at 10,000 cells per chamber. For immunofluorescence, the colonies in Matrigel were fixed with 2% freshly prepared paraformadehyde and analyzed using a BioRad confocal microscope, as described (Debnath et al, 2002; Ryo et al., 2002). For histology, the fixed colonies were paraffin-embedded and processed like tissue blocks. Antibodies used were anti-Ecadherin (Becton), Rat anti Ki67 (Dako) and Rat anti alpha 6 integrin (G0H3, Chemicon).

Retroviral Gene Transfer

Cyclin Dl and cyclin Dl 286A in pBabe were a gift from Drs. J. Debnath and J. Brugge. Murine cyclin Dl and its constitutively active mutant cyclin Dl 286A were subcloned into the retroviral vector WIRES from Dr. A. M. Kenney, in which Blasticidin resistance sequence had been replaced with GFP. The constructs were co-transfected with VSV and gag-pol into the packaging cell line 293 EBNA as described Debnath, 2002 #2184]. The primary MECs were infected on three consecutive days for 6 hours each. On day 4 they were subjected to 3D culture assay.

Tumorigenicity Assay

100,000 primary MECs isolated from Neu Pinl+/+ or Neu/Pin−/− mice were subjected to 3D cultures for 21 days. All developing structures were harvested and resuspended in 100]A MEGM/4% Matrigel. They were injected subcutaneously under the back skin of 5- to 6-week-old NCr athymic female nude mice (Taconic), in duplicates each (right and left flank). Mice were observed weekly for the visual appearance of tumors at injection sites.

Statistical Analysis

Nine cohorts were considered for the analysis of the endpoint, disease-free survival. The Kaplan-Meier method was used to estimate disease-free survival for each cohort. The significance of the differences in disease-free survival among the cohorts was determined with the use of log-rank (Mantel-Cox) test.

### Example 1

### PINl Deletion Mice Do Not Develop Breast Cancer when Over Expressing Ras or Neu

Pinl deletion mice have been previously generated and found to have no overt phenotype (Fujimori, et al., (1999) Biochem Biophys Res Commun 265, 658-663). It is shown in this example that Pinl−/− mice are largely protected from breast cancers induced by the Her2/Neu or Ras transgenes.

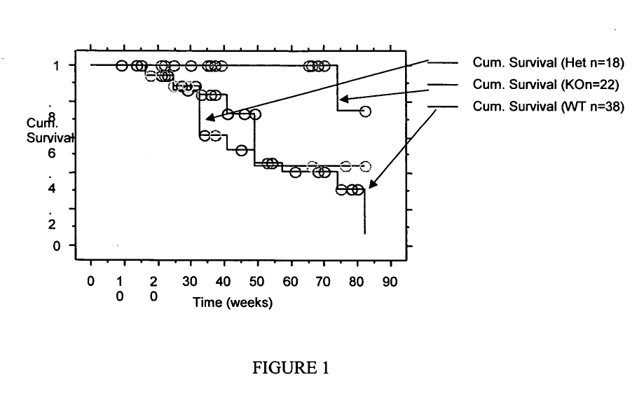

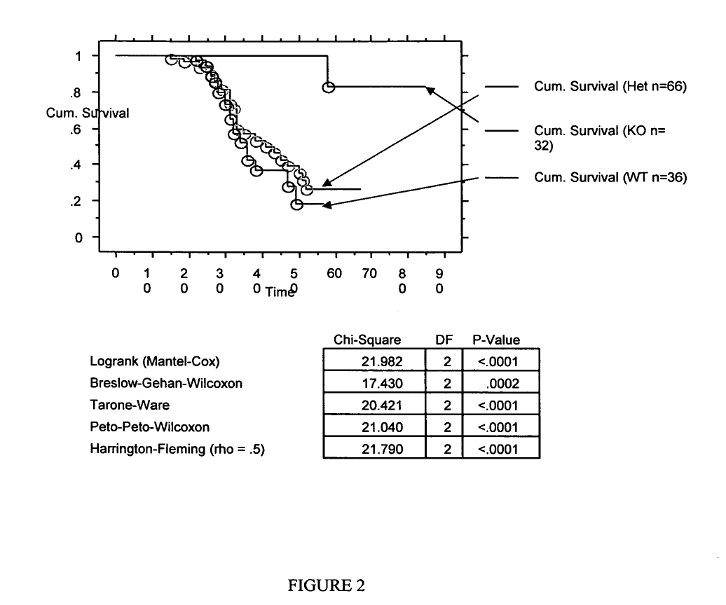

Specifically, it was determine whether the absence or presence of Pinl affected the incidence of breast cancer in MMTV-Ras or MMTV-Neu transgenic mice. Historically the MMTV-Ras or MMTV-Her2/Neu transgenic mice on an FVB background have developed tumors at a medium age of 14-16 weeks (Muller W, Sinn E, Pattengale P K, Wallace R and Leder P, Cell 54(105-115) 1988), and on a mixed background within a time range of 25-50 weeks (Yu, Q, Geng Y and Sicinski P, Nature 2001, 411(1017-1021). Mice with Pinl−/− mice were bred on a mixed 129/Sv and C57L/B6 background. Transgene positive F1 (Pinl heterozygote) mice were mated with transgene negative F1 (Pinl heterozygote) mice to generate F2. Transgene positive Pinl−/−, Pinl+/+ and Pinl± mice with this triple mixed background were enrolled in the study. The mice were kept as virgins and observed for 70 weeks, and their probability of disease-free survival was analyzed using the Kaplan-Meier method. Transgene positive Pinl+/+ mice developed tumors at a median age of 32 weeks (Her2/neu) (FIG. 1) or 49 weeks (Ras) (FIG. 2). Transgene positive Pinl−/− mice however were largely protected from the development of breast cancers to the extent that the probability of disease-free survival at age 70 weeks increased from 20 percent to over 80 percent (p<0.0002) for mice carrying the Her2/neu transgene, and from 20 to over 75 percent for Ras-transgenic mice (p<0.02). Thus, the absence of the Pinl gene largely protected these animals from breast cancers induced by the Her2/Neu or Ras transgene.

### Example 2

### Three Dimensional Primary Cell Differentiation Assay

To further confirm these above results, we have developed an in vitro culture system that allows one to predict the fate of individual mammary epithelial cells, specifically whether they develop normally or form breast cancers. Mammary epithelial cells were first isolated from mammary glands from mice. Female mice that carry the human breast cancer oncogene Her2/Neu were sacrificed using C02 narcosis, and the inguinal mammary glands were isolated in a sterile autopsy. The glands were mechanically disaggregated using surgical scissors and then resuspended in a medium that contains collagenase (ICN) at 1 mg/ml in a base of DMEM/F12 (Gibco). The flask was placed on a shaker at 37 C and shaken at 100 rpm for 3 hours. Then collagenase was neutralized with DMEM/F12/10% horse serum, and the suspension is centrifuged at 1200 rpm×10 min. The resulting pellet was washed with sterile HBSS twice. Then Trypsin EDTA (Gibco) was added and the culture is re-incubated for 15 min at 37 C. They were washed once with DMEM/F12/10% horse serum, and twice with HBSS. The resulting pellet was resuspended in MEGM media (Clontech) and the cells were grown for 5-7 days in a 10% C02 incubator with medium change every other day.

For the three-dimensional differentiation system the cells were trypsinized, counted and resuspended in MEGM at a concentration of 100,000 per milliliter. They were then mixed with a medium containing DMEM/F12, 4% Matrigel (BD Biosciences), Hydrocortisone (100 mcg/ml), Insulin (10 mcg/ml), Cholera toxin (1 ng/ml). The final concentration of EGF (Epidermal growth factor) was 5 ng/ml. The medium was replaced every 4 days. After 14-20 days in culture the following types of structures were derived from a morphologically normal mammary gland from a mouse carrying the Her2/Neu transgene: 1. Simple, well-organized structures that correspond to normal breast ducts; 2. Complex structures with partially filled lumina that correspond to Atypical Ductal Hyperplasia or Ductal Carcinoma in Situ; and 3. Complex structures that have an invasive growth pattern and infiltrate the matrigel base. These correspond to infiltrating carcinoma. All three structures were derived from mice that were transgenic for the human breast cancer oncogene Her2/neu, but that have not yet developed breast cancer. However, primary breast epithehal cells derived from Her2 transgenic mice with genetic Pinl deletion did not develop these transformed phenotypes.

### Example 3

### PINl Expression in Transgenic Mice

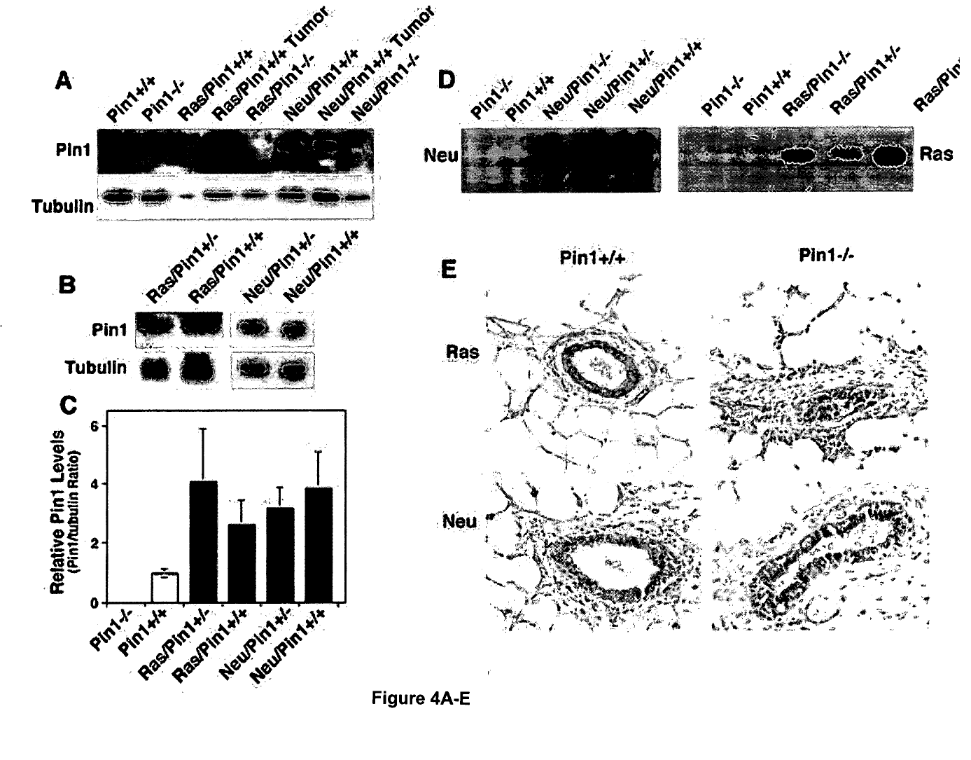

It has been demonstrated that Pinl is overexpressed in human breast cancer tissues and that Pinl expression is increased by activated Neu or Ras (Ryo et al, 2002; Ryo et al, 2001; Wulf et al, 2001). To examine the role of Pinl in breast cancer induced by Neu and Ras, Pinl knockout (Pinl−/−) mice (Liou et al, 2002) and oncogenic transgenic mice overexpressing an activated rat Neu/Her2/ErbB2 kinase (c-Neu) or v-Ha-Ras under the control of the MMTV promoter were crossed (Bouchard et al, 1989; Muller et al., 1988; Sinn et al., 1987). As compared with normal controls, Pinl levels were consistently increased several-fold in mammary glands or mammary tumors isolated from Neu/Pinl+/+ or Ras/Pinl+/+ animals (FIGS. 4A, C). However, no Pinl protein was detected in mammary gland lysates in all Pinl−/− mice regardless of the transgene (FIGS. 4A, C). No significant difference in Pinl levels between Pinl+/+ and Pinl± mice (FIG. 4B) was found. These results indicate that Pinl protein is absent in Pinl−/− mice, but remains at wild-type levels in Pinl± mice.

### Example 4

### PINL Ablation Affects Neither the Development of Virgin Mammary Glands nor the Expression of Transgenes

This example investigates the effects of Pinl ablation on the oncogenic processes. It has been reported that mammary glands in Pinl−/− or MMTV-Neu or -Ras transgenic virgin females develop normally (Liou et al, 2002; Yu et al, 2001), although Pinl−/− mammary glands fail to undergo the massive proliferation during pregnancy (Liou et al., 2002). To address the question whether the combination of the transgene with Pinl deletion affected mammary gland development, whole mount and histological analyses was performed (Liou et al., 2002; Yu et al., 2001). Morphometric analysis of carmine-stained whole-mounts of the virgin mammary glands revealed inter-individual variations, but no significant difference in the number of primary ducts, secondary branches or end buds between Pinl+/+ and Pinl−/− mice carrying the Ras or Neu transgene (Table 1 and Table 2). All virgin female mice developed proper mammary ducts with an intact lumen and again there was no detectable difference between Pinl+/+ and Pinl−/− background.

It was next investigated whether Pinl ablation could affect the expression of the c-Neu or Ha-Ras transgene. It has been shown that expression levels of these transgenes are typically low in non-neoplastic mammary glands, although they tend to be much higher in mammary tumors (Muller et al, 1988, Bouchard, 1989 #2245; Sinn et al, 1987; Yu et al, 2001). In addition, the transgenes are only expressed in MECs, not in the surrounding architectural and fat pad tissue, which make up for the bulk of the mammary gland in the virgin mouse (FIG. 4E). Immunohistochemistry and immunoblotting analyses were used to detect the expression of the c-Neu or Ha-Ras transgene. Both assays showed no detectable difference in transgene expression in mammary glands between Pinl+/+ and Pinl−/− mice (FIGS. 4E, F). These results indicate that Pinl ablation does not affect the expression of the transgenes.

### Example 5

### PINL Ablation Effectively Blocks the Induction of Cyclin DL by Neu or Ras

It has been shown that in Neu- or Ras-transgenic mice, cyclin Dl is induced, which is essential for Neu or Ras-induced breast cancer (Yu et al., 2001). It had previously been shown that Pinl positively regulates cyclin Dl levels by transcriptional activation and post-translation stabilization in response to growth signals in vitro (Liou et al., 2002; Ryo et al., 2001; Wulf et al., 2001). These results suggest that loss of Pinl might block the induction of cyclin Dl in Neu- or Ras-transgenic mice. Therefore, cyclin Dl expression in mammary glands derived from different genetically modified mice by was analyzed by immunoprecipitation, followed by immunoblotting analysis with anti-cyclin Dl antibodies.

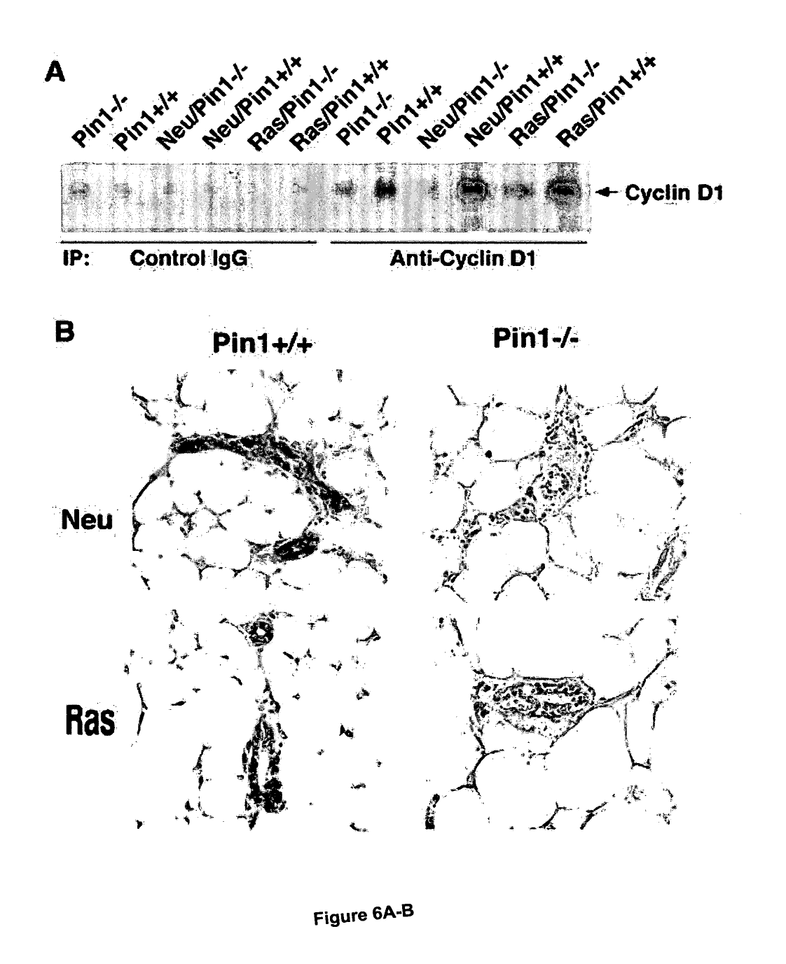

As shown (Liou et al., 2002; Yu et al., 2001), cyclin Dl was lower in Pinl−/− mice, but induced in Neu or Ras transgenic mice in the Pinl+/+ genetic background (FIG. 6A). However, in the Pinl−/− genetic background, cyclin Dl was barely induced in Neu or Ras transgenic mice (FIG. 6A). To confirm these results, immunohistochemistry was performed using anti-cyclin Dl antibodies. While cyclin Dl immunostaining signals were readily detected in MECs in Neu/Pinl+/+ or Ras/Pinl+/+, there was barely and detectable cyclin Dl signals in Neu/Pinl−/− or Ras/Pinl−/− mice (FIG. 6B). These results indicate that Pinl ablation effectively blocks the induction of cyclin Dl by Neu or Ras.

### Example 6

### PINL Ablation does not Affect the Differentiation of Primary Mouse Mammary Epithelial Cells (MECS) in 3 Dimensional (3D) Cultures

Pinl ablation is effective in suppressing breast cancer induced by Neu or Ras. Therefore a system was established with ex vivo cultures of primary MECs derived from Pinl ablated mice to determine whether Pinl deletion affects the growth and differentiation properties of mammary epithelial cells (MECs).

Primary MECs were isolated from morphologically normal mammary glands of wild-type mice or Neu or Ras transgenic mice in Pinl+/+ or Pinl−/− background at ages of 3-4 months. Histological examinations of the inguinal mammary gland that was contralateral to the mammary gland used for ex vivo cultures were performed to examine the possibility that small microscopic foci of tumors that were macroscopically not yet detectable might affect the ex vivo culture. No invasive or in situ carcinoma was detected at these early stages. Furthermore, no significant difference among these different genetic backgrounds when primary MECs were cultured on collagen-coated dishes (2D cultures). All cells appeared as a rather homogenous population that grew in an anchorage-dependent fashion, required growth factor for survival, and eventually stopped growing within 2 weeks ex vivo.

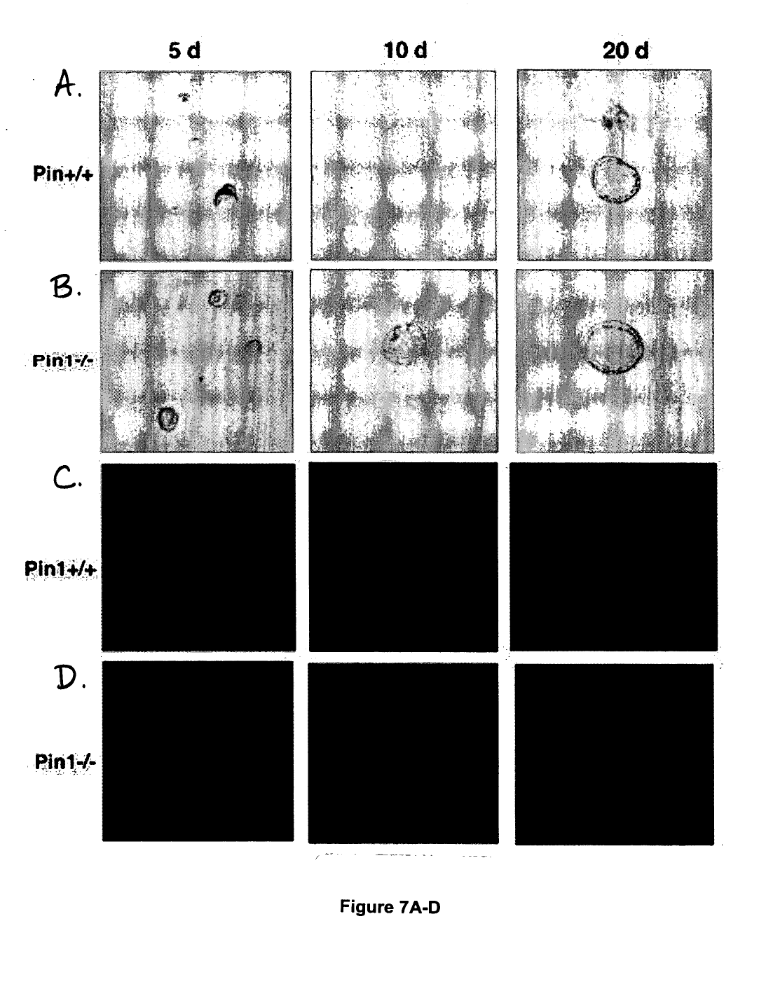

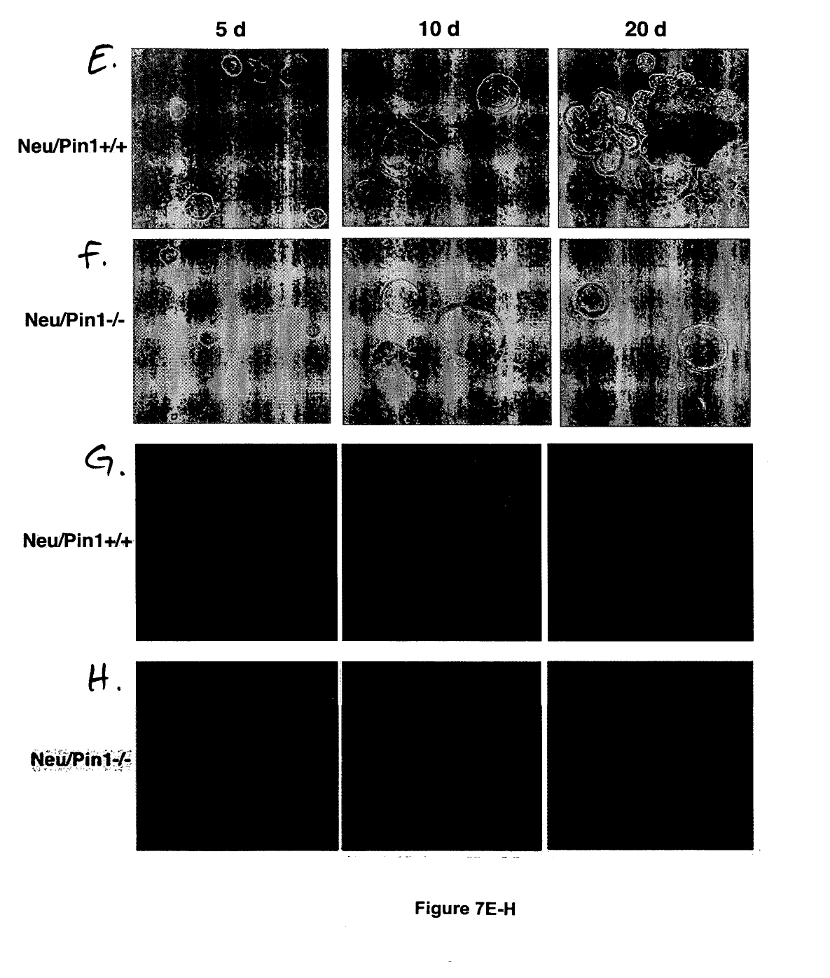

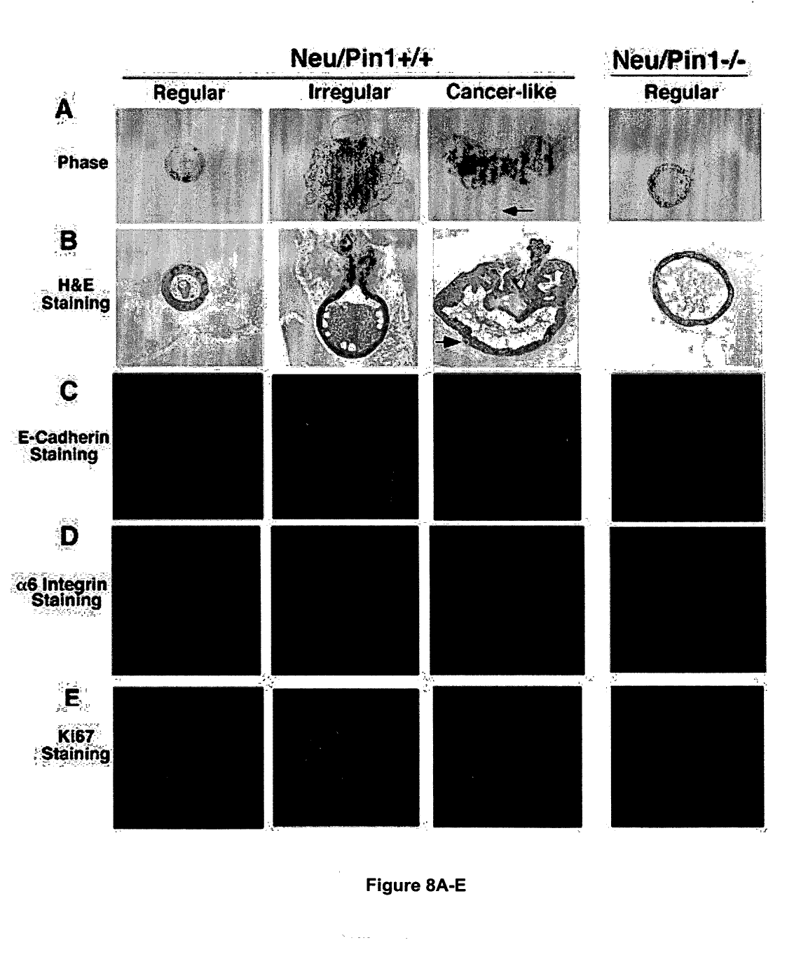

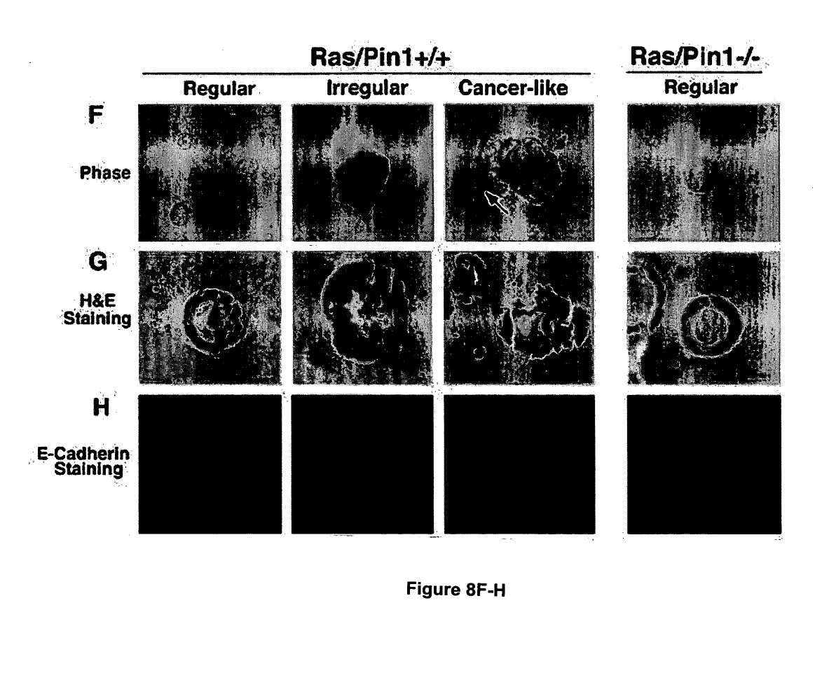

Primary MECs were plated as single cell suspension in reconstituted basement membrane using modified culture media (3D cultures). MECs from Pinl+/+ or Pinl−/− mice began to form globular colonies, and the cells in the center started to undergo apoptosis. These globular colonies then developed into organized and polarized acinus-like colonies with an intact lumen by day 10, followed by a stop in cell growth by day 20 of cultures (FIG. 7A). These orderly differentiated “Regular” colonies exhibited polarized expression of E-cadherin (FIG. 7A) and showed lost or low-level Ki67 expression (FIG. 8E). These in vitro differentiation patterns are similar to those described of human primary MECs and normal MEC cell line MCFIOA (Debnath et al., 2002; Gudjonsson et al., 2002). They indicate that the deletion of Pinl does not affect orderly and terminal differentiation of primary MECs ex vivo.

### Example 6

### Primary MECs of Neu or Ras Mice Display Various Malignant Properties, Including Forming Tumors in Nude Mice, Long Before they Develop Tumors in vivo

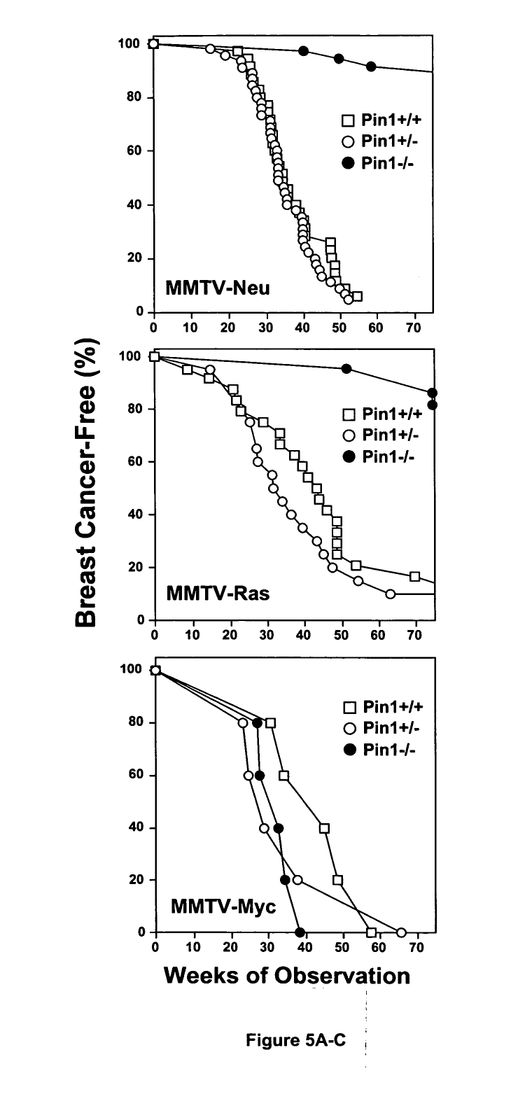

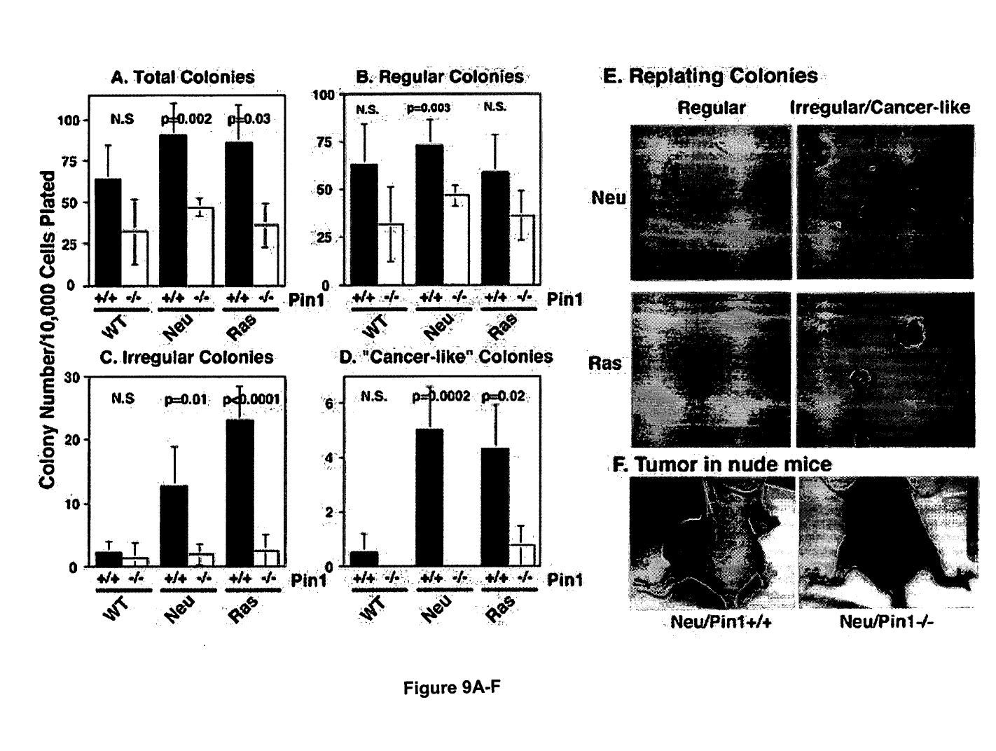

Distinct and strikingly different differentiation patterns for MECs derived from Neu or Ras transgenic (as opposed to wild-type) mice were observed (FIG. 7 and 8), although there were considerable inter-individual variations (FIG. 9A-D). Neu and Ras MECs tended to have an overall higher plating efficiency and higher colony counts than non-transgenic cells (FIG. 9A), suggesting that Ras and Neu transgenic animals may have an expanded MEC progenitor cell pool. The majority of primary MECs differentiate into well-differentiated round acinar colonies (FIG. 8 and 9B), as is the case for almost all cells derived from wild-type mice (FIG. 8A, F, first panel, 9B “Regular”). However, the stochastic, independent emergence of large, multi-acinar colonies with lumen filled were observed, which were rarely observed in non-transgenic MECs (FIG. 8A, F, second panel, 9C “Irregular”). More interestingly, we also observed expansive colonies with invading cells emerging from the original acinar colonies (FIG. 8A, F, third panel, 9D). These “Cancer-like” colonies were reproducibly observed in all primary MEC cultures derived from Neu or Ras transgenic mice, but not from any non-transgenic mice (FIG. 8). H&E staining showed that the “Regular” colonies were formed by uniform MECs with basally polarized nuclear organization, small nuclei and abundant cytoplasm (FIG. 8B, G). “Irregular” colonies were large, often had multiple acini, and their lumia were characteristically filled (FIG. 5B, G). “Cancer-like” colonies had disrupted cell polarity, cell surface spikes invading into the Matrigel, persistent mitotic figures, large and irregular nuclei, and high nuclear/cytoplasmic ratio (FIG. 5B, G).

Loss of E-cadherin expression, breaching of the basement membrane and continuous cell proliferation are some features of breast cancer cells (D'Ardenne et al., 1991; Moll et al., 1993; Pavelic et al., 1992). Therefore, immunofluorescence staining in situ was performed on these colonies with antibodies against E-cadherin, α6 integrins and Ki67. Consistent with the histological features, orderly and mostly basal expression of E-cadherin in the “Regular” colonies was observed (FIG. 8C, H). E-cadherin expression was lost in those cells that filled the lumen in “Irregular” colonies and even more obviously in “Cancer-like” colonies (FIG. 8C, H). Furthermore, “Regular” acini had the orderly, basal α6 integrin expression encircling the acini fully (FIG. 8D), a characteristic of normal mammary epithelial acini (D'Ardenne et al., 1991). This was in sharp contrast to disorganized a6 integrin expression in “Cancer-like” acini, where basal a6 integrin expression pattern was completely disrupted and epithelial cells broke through and invaded into the basal membrane-containing Matrigel (FIG. 8D). Moreover, “Irregular” and “Cancer-like” colonies continued to express Ki67 beyond day 20 of culture, while “Regular” acini tended to be Ki67-negative (FIG. 8E). These results together indicate that a significant fraction of primary Neu or Ras MECs fail to differentiate, but continuously grow into invasive colonies. Interestingly, these abnormal properties resemble to those of the normal MECs MCFIOA transformed with oncogenes in vitro (Debnath et al, 2002; Muthuswamy et al, 2001).

To further confirm that the “Regular” colonies are mostly composed of non-dividing terminally differentiated cells and the “Irregular” colonies contain actively dividing cells, “Regular” and “Irregular” colonies were isolated separately at day 21 and assayed for secondary colony formation. Although cells derived from “Regular” colonies gave rise to only very few “Regular” secondary acinar colonies, “Irregular” colonies gave rise to multiple “Irregular” colonies. These results indicate that the cells in these “Irregular” colonies retain their proliferative capacity, and that these colonies are indeed the result of clonal expansion of a distinct type of MECs (FIG. 9E).

Finally, to confirm that these “Cancer-like” colonies indeed contain cancer cells, they colonies were surgically transplanted into nude mice to examine their ability to form tumors. 1-2 months later after the transplantation, tumors were visually identified at 50% of sites that were transplanted with “Cancer-like” colonies formed by MECs derived from Neu transgenic mice, but not from control MECs (FIG. 9F). These results indicate that a significant fraction of primary MECs derived from morphologically and histologically normal mammary gland of Neu or Ras mice exhibit the malignant phenotype ex vivo.

### Example 7

### Ablation of Pinl Suppresses Early Transformed Properties of Neu or Ras MECs

Primary MECs derived from Neu- or Ras-transgenic mice display the transformed phenotype ex vivo long before they produce tumors in vivo. Therefore, it was investigated whether this transformed phenotype is affected by Pinl ablation. Like wild-type cells, Neu/Pinl−/− MECs and Ras/Pinl−/− MECs tended to have lower colony counts than their Pinl+/+ counterparts (FIG. 9A), indicating that loss of Pinl function may prevent the increase in the MEC progenitor cells seen in Neu or Ras transgenic mice. The frequency of “Irregular” colonies was greatly reduced in Neu/Pinl−/− or Ras/Pinl−/− MECs, as compared those from Neu/Pinl+/+or Ras/Pinl+/+ cells (FIG. 8, 9C). Furthermore, “Cancer-like” colonies were absent from Neu/Pinl−/− derived cultures and very rare in Ras/Pinl−/− cultures (FIG. 8, 9D). Moreover, colonies derived from Neu/Pinl−/− MECs failed to form any tumors when transplanted into nude mice (FIG. 9F). These data indicate that Pinl ablation effectively suppresses the early transformed phenotype of Ras or Neu MECs ex vivo.

### Example 8

### Overexpression of Cyclin Dl in Neu/Pinl−/− Primary MECs Rescues Their Malignant Phenotype

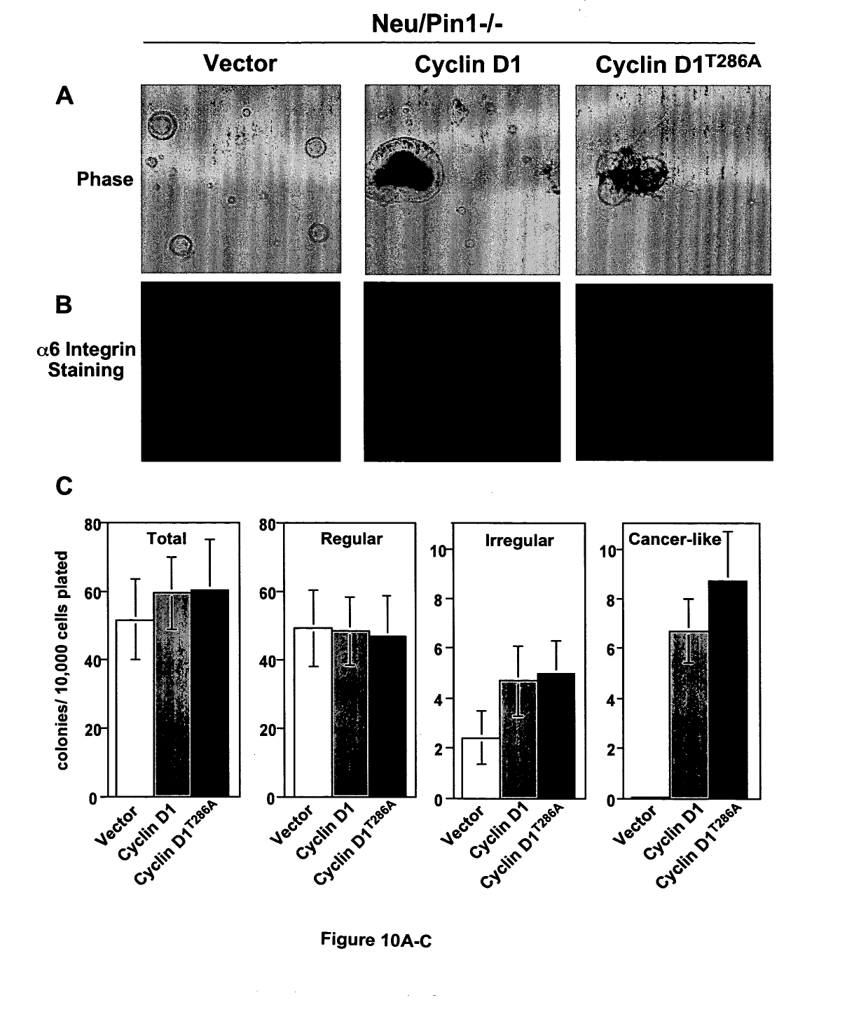

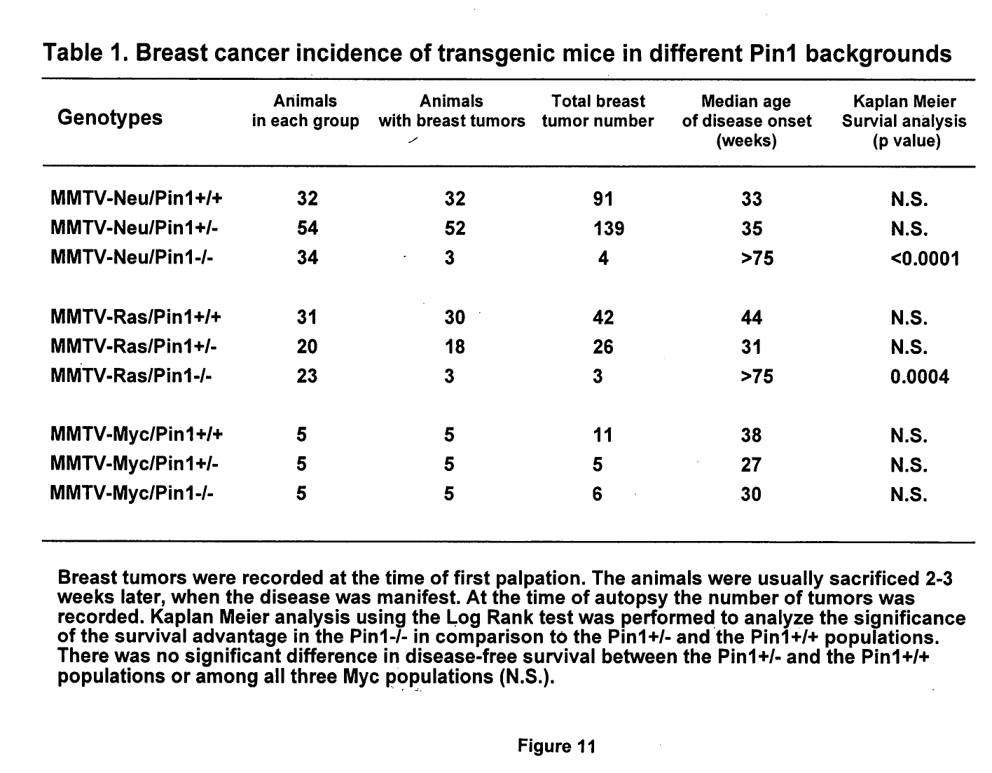

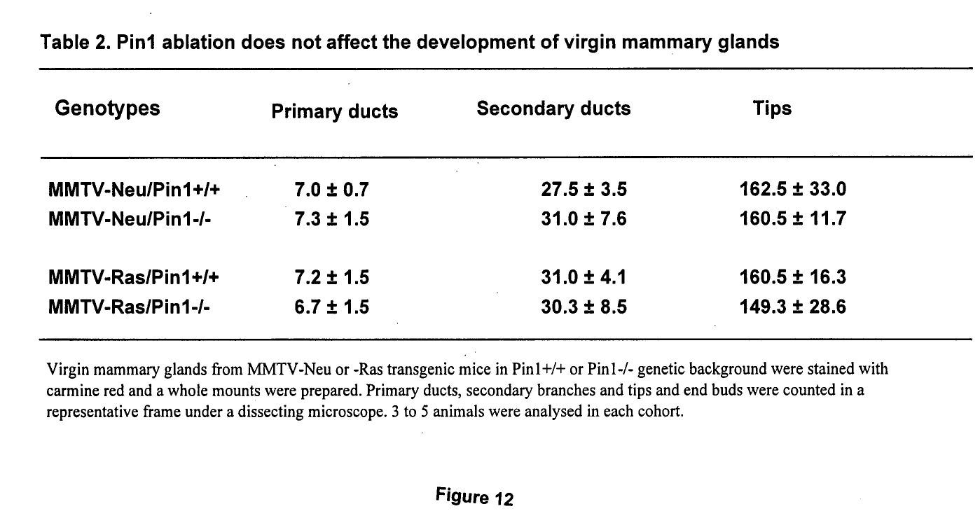

The above results indicate that the Pinl−/− genetic background, Neu or Ras fails to transform MEC and to induce breast cancer as well as to increase cyclin Dl expression. Since cyclin Dl is essential for Neu or Ras to induce breast cancer (Bowe et al., 2002; Yu et al., 2001), it was investigated whether the failure of Neu or Ras to induce cell transformation and breast cancer in the Pinl−/− genetic background is due to the absence of cyclin Dl induction. To address this question, retroviral gene transfer was used with concomitant GFP expression to deliver cyclin Dl or its T286A mutant to primary MECs derived from Neu/Pinl+/+ mice, as described (Debnath et al., 2002). Based on GFP expression, the infection efficiency was over 80% and transgene expression was confirmed by immunoblot. Importantly, when infected with cyclin Dl but not the control vector, Neu/Pinl−/− MECs generated “Cancer-like” colonies (FIG. 10A), with a similar incidence than Neu/Pinl+/+ cells (FIG. 10C, 9D). This “Cancer-like” phenotype was even more obvious when infected with the cyclin Dl^{T286A} mutant (FIG. 10B, C), a mutant known to be more stable and potent in transforming cells (Alt et al., 2000). These results further support the conclusion that the inhibition of tumorigenesis by Pinl ablation is due to the suppression of cyclin Dl.

### REFERENCES

Alt, J R, Cleveland, J L, Hannink, M and Diehl, J A (2000) Phosphorylation-dependent regulation of cyclin Dl nuclear export and cyclin Dl-dependent cellular transformation. Genes Dev 14: 3102-3114.
Arber, N, Doki, Y, Han, E K, Sgambato, A, Zhou, P, Kim, N H, Delohery, T, Klein, M G, Holt, P R and Weinstein, I B (1997) Antisense to cyclin Dl inhibits the growth and tumorigenicity of human colon cancer cells. Cancer Res 57: 1569-1574
Ayala, G, Wang, D, Wulf, G, Frolov, A, Le, R, Wheeler, T, Sowadski, J M, Lu, K P and Bao, L (2003) Pinl is a novel prognostic marker in prostate cancer. Cancer Research 63: 6244-6251
Bao, L, Sauter, G, Sowadski, J, Lu, K P and Wang, D (2004) Prevalent overexpression of prolyl isomerase Pinl in human cancers. Am. J. Pathol 164: 1727-1737
Barcellos-Hoff, M H, Aggeler, J, Ram, T G and Bissell, M J (1989) Functional differentiation and alveolar morphogenesis of primary mammary cultures on reconstituted basement membrane. Development 105: 223-235.
Blume-Jensen, P and Hunter, T (2001) Oncogenic kinase signaling. Nature 411: 355-365 Bouchard, L, Lamarre, L, Tremblay, P J and Jolicoeur, P (1989) Stochastic appearance of mammary tumors in transgenic mice carrying the MMTV/c-neu oncogene. Cell 57: 931-936.
Bowe, D B, Kenney, N J, Adereth, Y and Maroulakou, I G (2002) Suppression of Neu-induced mammary tumor growth in cyclin Dl deficient mice is compensated for by cyclin E. Oncogene 21: 291-298.
D'Ardenne, A J, Richman, P I, Horton, M A, McAulay, A E and Jordan, S (1991) Coordinate expression of the alpha-6 integrin laminin receptor sub-unit and laminin in breast cancer. JPathol 165: 213-220.
Debnath, J, Mills, K R, Collins, N L, Reginato, M J, Muthuswamy, S K and Brugge, (2002) The role of apoptosis in creating and maintaining luminal space within normal and oncogene-expressing mammary acini. Cell 111: 29-40.
Diehl, J A, Cheng, M, Roussel, M F and Sherr, C J (1998) Glycogen synthase kinase-3beta regulates cyclin Dl proteolysis and subcellular localization. Genes Dev 12: 3499-3511.
Diehl, J A, Zindy, F and Sherr, C J (1997) Inhibition of cyclin Dl phosphorylation on threonine-286 prevents its rapid degradation via the ubiquitin-proteasome pathway. Genes Dev 11: 957-972.
Fantl, V, Stamp, G, Andrews, A, Rosewell, I and Dickson, C (1995) Mice lacking cyclin Dl are small and show defects in eye and mammary gland development. Genes Dev 9: 2364-2372
Gillett, C, Fantl, V, Smith, R, Fisher, C, Bartek, J, Dickson, C, Barnes, D and Peters, G (1994) Amplification and overexpression of cyclin Dl in breast cancer detected by immunohistochemical staining. Cancer Res 54: 1812-1817
Gudjonsson, T, Ronnov-Jessen, L, Villadsen, R, Rank, F, Bissell, M J and Petersen, O W (2002) Normal and tumor-derived myoepithelial cells differ in their ability to interact with luminal breast epithelial cells for polarity and basement membrane deposition. J Cell Sci 115: 39-50.
Hanahan, D and Weinberg, R A (2000) The hallmarks of cancer. Cell 100: 57-70
Hinds, P W, Dowdy, S F, Eaton, E N, Arnold, A and Weinberg, R A (1994) Function of a human cyclin gene as an oncogene. Proc Natl Acad Sci USA 91: 709-713
J-.ee, R I, Albanese, C, Fu, M, D'Amico, M, Lin, B, Watanabe, G, Haines, G K, 3rd, Siegel, P M, Hung, M C, Yarden, Y, Horowitz, J M, Muller, W J and Pestell, R G (2000) Cyclin Dl is required for transformation by activated Neu and is induced through an E2F-dependent signaling pathway. Mol Cell Biol 20: 672-683
Liou, Y C, Ryo, R, Huang, H K, Lu, P J, Branson, R, Fujimori, F, Uchidafl, U, Hunter, T and Lu, K P (2002) Loss of Pinl function in the mouse causes phenotypes resembling cyclin Dl-null phenotypes. Proc. Natl. Acad. Sci. USA 99: 1335-1340
Lowenberg, B, van Putten, W L, Touw, I P, Delwel, R and Santini, V (1993) Autonomous proliferation of leukemic cells in vitro as a determinant of prognosis in adult acute myeloid leukemia. N Engl J Med 328: 614-619.
Lu, K P (2003) Prolyl isomerase Pinl as a molecular target for cancer diagnostics and therapeutics. Cancer Cell 4: 175-180
Lu, K P (2004) Pinning down cell signaling, cancer and Alzheimer's disease. TiBS 29: 200-209 Lu, K P, Hanes, S D and Hunter, T (1996) A human peptidyl-prolyl isomerase essential for regulation of mitosis. Nature 380: 544-547
Lu, K P, Liou, Y C and Zhou, X Z (2002) Pinning down the proline-directed phosphorylation signaling. Trends CellBiol 12: 164-172 Lu, P J, Zhou, X Z, Shen, M and Lu, K P (1999) A function of WW domains as phosphoserine- or phosphothreonine-binding modules. Science 283: 1325-1328
Moll, R, Mitze, M, Frixen, U H and Birchmeier, W (1993) Differential loss of E-cadherin expression in infiltrating ductal and lobular breast carcinomas. AmJ Pathol 143: 1731-1742.
Muller, W J, Sinn, E, Pattengale, P K, Wallace, R and Leder, P (1988) Single-step induction of mammary adenocarcinoma in transgenic mice bearing the activated c-neu oncogene. Cell 54:105-115.
Muthuswamy, S K, Li, D, Lelievre, S, Bissell, M J and Brugge, J S (2001) ErbB2, but not ErbB1, reinitiates proliferation and induces luminal repopulation in epithelial acini. Nat Cell Biol 3:785-792.
O'Brien, L E (2002) Opinion: Building epithelial architecture: insights from three-dimensional culture models. Nat Rev Mol Cell Biol 3: 531-537.
Pavelic, Z P, Pavelic, L, Lower, E E, Gapany, M, Gapany, S, Barker, E A and Preisler, H D (1992) c-myc, c-erbB-2, and Ki-67 expression in normal breast tissue and in invasive and noninvasive breast carcinoma. Cancer Res 52: 2597-2602.
Petersen, O W, Ronnov-Jessen, L, Howlett, A R and Bissell, M J (1992) Interaction with basement membrane serves to rapidly distinguish growth and differentiation pattern of normal and malignant human breast epithelial cells. Proc Natl Acad Sci USAS 9: 9064-9068.
Ryo, A, Liou, Y C, Lu, K P and Wulf, G (2003a) Prolyl isomerase Pinl: a catalyst for oncogenesis and a potential therapeutic target in cancer. J Cell Sci 116: 773-783
Ryo, A, Liou, Y C, Wulf, G, Nakamura, N, Lee, S W and Lu, K P (2002) Pinl is an E2F target gene essential for the Neu/Ras-induced transformation of mammary epithelial cells. Mol Cell Biol 22: 5281-5295
Ryo, A, Nakamura, N, Wulf, G, Liou, Y C and Lu, K P (2001) Pinl regulates turnover and subcellular localization of beta-catenin by inhibiting its interaction with APC. Nature Cell Biol 3: 793-801
Ryo, A, Suizu, F, Yoshida, Y, Perrem, K, Liou, Y C, Wulf, G, Rottapel, R, Yamaoka, S and Lu, K P (2003b) Regulation of NF-kappaB signaling by Pinl-dependent prolyl isomerization and ubiquitin-mediated proteolysis of p65/RelA. Mol Cell 12: 1413-1426
Senn, J S, McCulloch, E A and Till, J E (1967) Comparison of colony-forming ability of normal and leukaemic human marrow in cell culture. Lancet 2: 597-598.
Sicinski, P, Donaher, J L, Parker, S B, Li, T, Fazeli, A, Gardner, H, Haslam, S Z, Branson, R T, Elledge, S J and Weinberg, R A (1995) Cyclin Dl provides a link between development and oncogenesis in the retina and breast. Cell 82: 621-630
Sinn, E, Muller, W, Pattengale, P,.Tepler, I, Wallace, R and Leder, P (1987) Coexpression of MMTV/v-Ha-ras and MMTV/c-myc genes in transgenic mice: synergistic action of oncogenes in vivo. Cell 49: 465-475.
Smalley, M and Ashworth, A (2003) Stem cells and breast cancer: A field in transit. Nat RevCancer 3: 832-844.
Wang, T C, Cardiff, R D, Zukerberg, L, Lees, E, Arnold, A and Schmidt, E V (1994) Mammary hyperplasia and carcinoma in MMTV-cyclin Dl transgenic mice. Nature 369: 669-671.
Wulf, G M, Liou, Y C, Ryo, A, W., L S and Lu, K P (2002) Role of Pinl in the regulation of p53 stability and p21 transactivation, and cell cycle checkpoints in response to DNA damage. J Biol Chem 277: 47976-47979
Wulf, G M, Ryo, A, Wulf, G G, Lee, S W, Niu, T and Lu, K P (2001) Pinl is overexpressed in breast cancer and potentiates the transcriptional activity of phosphorylated c-Jun towards the cyclin Dl gene. EMBO J 20: 3459-3472
Yaffe, M B, Schutkowski, M, Shen, M, Zhou, X Z, Stukenberg, P T, Rahfeld, J, Xu, J, Kuang, J, Kirschner, M W, Fischer, G, Cantley, L C and Lu, K P (1997) Sequence-specific and phosphorylation-dependent proline isomerization: A potential mitotic regulatory mechanism. Science 278: 1957-1960
Yeh, E, Cunningham, M, Arnold, H, Chasse, D, Monteith, T, Ivaldi, G, Hahn, W C, Stukenberg, P T, Shenolikar, S, Uchida, T, Counter, C M, Nevins, J R, Means, A R and Sears, R (2004) A signalling pathway controlling c-Myc degradation that impacts oncogenic transformation of human cells. Nat Cell Biol 6: 308-318
Yu, Q, Geng, Y and Sicinski, P (2001) Specific protection against breast cancers by cyclin Dl ablation. Nature 411: 1017-1021
Zacchi, P, Gostissa, M, Uchida, T, Salvagno, C, Avolio, A, Voliniak, S, Ronai, Z, Blandino, G, Schneider, C and Del Sal, G (2002) The prolyl isomerase Pinl reveals a mechanism to control p53 functions after genotoxic insults. Nature 419: 853-857
Zheng, H, You, H, Zhou, X Z, Murray, S A, Uchida, T, Wulf, G, Gu, L, Tang, X, Lu, K P and Xiao, Z X J (2002) The prolyl isomerase Pinl is a regulator of p53 in genotoxic response. Nature 419: 849-853
Zhou, X Z, Kops, O, Werner, A, Lu, P J, Shen, M, Stoller, G, Kiillertz, G, Stark, M, Fischer, G and Lu, K P (2000) Pinl-dependent prolyl isomerization regulates dephosphorylation of Cdc25C and tau proteins. Mol Cell 6: 873-883

1. A method of determining if a subject will benefit from treatment with a Pinl inhibitor comprising the steps of:
obtaining a biological sample from said subject; and
evaluating said biological sample for the presence of a cancer associated polypeptide;
wherein the presence the cancer associated polypeptide indicates that the subject will benefit from treatment with a Pinl inhibitor.

2. The method of claim 1, wherein said biological sample is from a tumor.

3. The method of claim 1, wherein said cancer associated polypeptide is encoded by an oncogene.

4. The method of claim 1 wherein said cancer associated polypeptide is selected from the group consisting of: her2/neu, ras, cyclin Dl, Cdk4, E2F, Myc, Jun, and Rb.

5. The method of claim 1 wherein said subject has cancer.

6. The method of claim 5 wherein said cancer is selected from the group consisting of:

7. The method of claim 6, wherein said cancer is breast cancer.

8. The method of claim 1 wherein said cancer associated peptide is misexpressed when compared to a control sample.

9. The method of claim 1, wherein said cancer associated polypeptide is her2/neu.

10. The method of claim 9, wherein said her2/neu is misexpressed when compared to a control sample.

11. The method of claim 1, wherein said cancer associated polypeptide is ras.

12. The method of claim 11, wherein said ras is misexpressed when compared to a control sample.

13. A method for determining if a subject will benefit from treatment with a cancer associated polypeptide inhibitor comprising the steps of:
obtaining a biological sample from said subject; and
evaluating said biological sample for the concentration of Pinl;
wherein an elevated concentration of Pinl in the biological sample indicates that the subject will benefit from treatment with a cancer associated polypeptide inhibitor.

14. The method of claim 13, wherein said Pinl concentration is phosphorylated Pinl.

15. The method of claim 13, wherein said Pinl concentration is unphosphorylated Pinl.

16. The method of claim 13, wherein the concentration of phosphorylated Pinl to unphosphorylated Pinl is determined.

17. The method of claim 13, wherein said biological sample is from a tumor.

18. The method of claim 13, wherein said cancer associated polypeptide is encoded by an oncogene.

19. The method of claim 13, wherein said cancer associated polypeptide is selected from the group consisting of: her2/neu, ras, cyclin Dl, Cdk4, E2F, Myc, Jun, and Rb.

20. The method of claim 13, wherein said subject has cancer.

21. The method of claim 20 wherein said cancer is selected from the group consisting of:

22. The method of claim 21, wherein said cancer is breast cancer.

23. The method of claim 13, wherein said Pinl is misexpressed when compared to a control sample.

24. The method of claim 13, wherein said cancer associated polypeptide is her2/neu.

25. The method of claim 24, wherein said her2/neu is misexpressed when compared to a control sample.

26. The method of claim 13, wherein said cancer associated polypeptide is ras.

27. The method of claim 26, wherein said ras is misexpressed when compared to a control sample.

28. A method of determining if a subject will benefit from treatment with a Pinl inhibitor in combination with a second cancer treatment comprising the steps of:
obtaining a biological sample from a subject; and
evaluating said biological sample for the presence of Pinl;
wherein the presence of Pinl is indicative that said subject will benefit from treatment with a Pinl inhibitor and a second cancer treatment specific for the cancer associated polypeptide.

29. The method of claim 28 further comprising evaluating said biological sample for the presence of a cancer associated polypeptide.

30. The method of claim 28, wherein said biological sample is from a tumor.

31. The method of claim 28, wherein said cancer associated polypeptide is encoded by an oncogene.

32. The method of claim 28, wherein said cancer associated polypeptide is selected from the group consisting of: her2/neu, ras, cyclin Dl, Cdk4, E2F, Myc, Jun, and Rb.

33. The method of claim 28, wherein said subject has cancer.

34. The method of claim 33, wherein said cancer is breast cancer.

35. The method of claim 28, wherein said Pinl is misexpressed when compared to a control sample.

36. The method of claim 28, wherein said cancer associated polypeptide is her2/neu.

37. The method of claim 28, wherein said her2/neu is misexpressed when compared to a control sample.

38. The method of claim 28, wherein said cancer associated polypeptide is ras.

39. The method of claim 38, wherein said ras is misexpressed when compared to a control sample.

40. The method of claim 28, wherein said cancer associated polypeptide is her2/neu and said second cancer treatment is herceptin.

41. A method of treating a subject having a neoplasitic disorder associated with misexpression of a cancer-associated polypeptide comprising:
administering to said subject a Pinl inhibitor;
thereby treating said subject.

42. The method of claim 41, wherein said cancer associated polypeptide is an oncogene.

43. The method of claim 42, wherein said oncogene is her2/neu.

44. The method of claim 41, wherein said cancer associated polypeptide is selected from the group consisting of: her2/neu, ras, cyclin Dl, Cdk4, E2F, Myc, Jun, and Rb.

45. The method of claim 44, wherein said cancer associated polypeptide is her2/neu.

46. The method of claim 41, wherein said neoplasitic disorder associated with misexpression of a cancer-associated polypeptide is breast cancer.

47. The method of claim 45, wherein said subject is administered a her2/neu specific cancer treatment.

48. The method of claim 47, wherein said her2/neu specific cancer treatment is herceptin.

49. A method of treating a subject resistant to a first cancer therapy comprising:
administering to said subject a Pinl inhibitor;
thereby treating said subject.

50. The method of claim 49, wherein said subject resistant to a her2/neu specific cancer therapy.

51. The method of claim 50, wherein said her2/neu specific cancer therapy is herceptin.

52. A method of treating a subject having a tumor that expresses Pinl and a cancer associated gene comprising;
administering to said subject a Pinl inhibitor and a second cancer therapy thereby treating said subject having a tumor.

53. The method of claim 52, wherein said Pinl inhibitor and said second cancer therapy are administered in quantities different than the quantity that is necessary to be effective if administered alone.

54. The method of claim 52 wherein said quantity is lower than is necessary to be effective alone.

55. The method of claim 52, wherein said second cancer therapy is herceptin.

56. The method of claim 52 wherein said subject has breast cancer.

57. An animal model for Pinl-related diseases comprising; a transgenic mouse expressing a cancer associated polypeptide that is Pinl−/−.

58. The animal model of claim 57 wherein said animal is a mammal.

59. The animal model of claim 58 wherein said animal is a mouse.

60. The animal model of claim 57 wherein said cancer associated gene is an oncogene.

61. The animal model of claim 60, wherein said oncogene is selected from the group consisting of: her2/neu, ras, cyclin Dl, Cdk4, E2F, Myc, Jun, and Rb.

62. A method of determining the invasive potential of a primary pre-malignant cell comprising;
obtaining a biological sample from a subject;
isolating cells of interest;
growing said cells on a membrane matrix; and
analyzing type of growth to thereby determining if a cell has invasive potential.

63. The method of claim 62, wherein said cell is an epithelial cell.

64. The method of claim 63, wherein said epithelial cell is isolated from breast tissue.

65. The method of claim 64 wherein said cell grows invasively into the membrane matrix.

66. The method of claim 65, wherein said invasive growth is characteristic of a cell developing into an infiltrating carcinoma.
